SEQUENCE LISTING (110 sequences)

SEQ ID NO: 1 - DNA, 2739 bp, Artificial Sequence
  Synthetic DNA
gggcgcgagctcggtacccggggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 2 - DNA, 2860 bp, Artificial Sequence
  Synthetic DNA
atggagctcaaagacatataacccacaagatatcgaacagccgctttacgagcactgggaaaagcagggctactttaagcctaatggcgatgaaagccaggaaagtttctgcatcatgatcccgccgccgaacgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctgtaa

SEQ ID NO: 3 - DNA, 1644 bp, Artificial Sequence
  Synthetic DNA
tagaatactcaagctttatgaccatcactccagaaacctctcgtccaatcgataccgaaagctggaaatcttactacaagtctgatccactgtgttccgctgtcctcatccatatgaaagaactgacccagcataacgttactccagaggacatgtctgcatttcgtagctaccagaaaaaactcgagctgtctgaaacctttcgcaaaaactactccctggaggacgaaatgatctactaccaagaccgtctcgtggttccgatcaagcaacaaaatgcggttatgcgtctgtaccatgatcatactctgttcggtggtcatttcggtgttactgttactctggctaaaatctctccgatctactattggccgaaactccagcactctatcatccagtacatccgtacctgcgttcagtgtcagctgatcaaatctcatcgtcctcgcctccacggtctcctccagcctctcccgatcgctgagggtcgttggcttgacattagcatggacttcgtgactggtttgccgccgacctctaataatcttaacatgatcctggttgtagtggaccgattctctaagcgtgctcacttcatcgcgactcgtaaaaccctggatgcaacccaattgatcgatctgctctttcgttacatcttctcctaccatggctttcctcgtaccatcacctctgatcgtgacgttcgtatgactgcggataaataccaggaactcaccaaacgtctgggtatcaaatctaccatgtctagcgcaaatcatccgcaaactgatggtcaatccgaacgtactatccaaaccctgaatcgtcttctgagggcgtatgctagcaccaacatccagaattggcacgtgtacctcccgcaaattgaattcgtgtacaattccactccgactcgcactctgggtaaatcaccattcgaaatagatctgggttatctgcctaatacacccgctatcaaatccgatgatgaagttaacgcgaggagcttcactgctgtagagctggctaaacatctgaaagccctgaccatccagaccaaagaacagcttgaacatgctcagattgaaatggaaaccaacaacaaccaacgtcgtaaacctctgctgttgaacatcggtgatcatgttctggttcatcgtgatgcatacttcaaaaagggtgcctacatgaaagttcagcagatttatgttggcccttttcgagtggtcaagaagatcaatgacaacgcatatgaactggacctgaactcccataaaaagaagcatcgtgtgatcaacgtgcaattcctgaaaaagtttgtttaccgtcctgatgcgtatcctaaaaacaaaccgatctcttctaccgagcgaatcaaacgcgcccatgaagttacggctctgataggtattgatactacccacaaaacctacctgtgtcacatgcaagacgttgacccgactctgtctgttgaatactctgaagccgaattctgtcagattccagagcgtacccgtcgttctatcctggctaattttcgtcaactgtacgagactcaggacaacccagaacgtgaggaagacgtcgtgagccaaaacgaaatctgccagtacgataacacctctccgaagcttgcatgcctgc

SEQ ID NO: 4 - DNA, 45 bp, Artificial Sequence
  Synthetic DNA
tagaatactcaagctttatgaccatcactccagaaacctctcgtc

SEQ ID NO: 5 - DNA, 44 bp, Artificial Sequence
  Synthetic DNA
gcaggcatgcaagcttcggagaggtgttatcgtactggcagatt

SEQ ID NO: 6 - DNA, 306 bp, Artificial Sequence
  Synthetic DNA
atgaccatcactccagaaacctctcgtccaatcgataccgaaagctggaaatccaatcgataccgaaagctggaaatcttactacaagtctgatccactgtgtcttactacaagtctgatccactgtgttccgctgtcctcatccatatgaaattccgctgtcctcatccatatgaaagaactgacccagcataacgttactccgaactgacccagcataacgttactccagaggacatgtctgcatttcgtagcagaggacatgtctgcatttcgtagctaccagaaaaaactcgagctgtctga

SEQ ID NO: 7 - DNA, 306 bp, Artificial Sequence
  Synthetic DNA
gaactgacccagcataacgttactccagaggacatgtctgcatttcgtagcagaggacatgtctgcatttcgtagctaccagaaaaaactcgagctgtctgataccagaaaaaactcgagctgtctgaaacctttcgcaaaaactactccctgaacctttcgcaaaaactactccctggaggacgaaatgatctactaccaagagaggacgaaatgatctactaccaagaccgtctcgtggttccgatcaagcaaccgtctcgtggttccgatcaagcaacaaaatgcggttatgcgtctgtacca

SEQ ID NO: 8 - DNA, 306 bp, Artificial Sequence
  Synthetic DNA
gaggacgaaatgatctactaccaagaccgtctcgtggttccgatcaagcaaccgtctcgtggttccgatcaagcaacaaaatgcggttatgcgtctgtaccacaaaatgcggttatgcgtctgtaccatgatcatactctgttcggtggtcattgatcatactctgttcggtggtcatttcggtgttactgttactctggctaattcggtgttactgttactctggctaaaatctctccgatctactattggccgaatctctccgatctactattggccgaaactccagcactctatcatccagta

SEQ ID NO: 9 - DNA, 306 bp, Artificial Sequence
  Synthetic DNA
ttcggtgttactgttactctggctaaaatctctccgatctactattggccgaatctctccgatctactattggccgaaactccagcactctatcatccagtaaaactccagcactctatcatccagtacatccgtacctgcgttcagtgtcagcatccgtacctgcgttcagtgtcagctgatcaaatctcatcgtcctcgcctctgatcaaatctcatcgtcctcgcctccacggtctcctccagcctctcccgccacggtctcctccagcctctcccgatcgctgagggtcgttggcttgacat

SEQ ID NO: 10 - DNA, 306 bp, Artificial Sequence
  Synthetic DNA
ctgatcaaatctcatcgtcctcgcctccacggtctcctccagcctctcccgccacggtctcctccagcctctcccgatcgctgagggtcgttggcttgacatatcgctgagggtcgttggcttgacattagcatggacttcgtgactggtttgtagcatggacttcgtgactggtttgccgccgacctctaataatcttaacatccgccgacctctaataatcttaacatgatcctggttgtagtggaccgattcgatcctggttgtagtggaccgattctctaagcgtgctcacttcatcgcgac

SEQ ID NO: 11 - DNA, 306 bp, Artificial Sequence
  Synthetic DNA
ccgccgacctctaataatcttaacatgatcctggttgtagtggaccgattcgatcctggttgtagtggaccgattctctaagcgtgctcacttcatcgcgactctaagcgtgctcacttcatcgcgactcgtaaaaccctggatgcaacccaatcgtaaaaccctggatgcaacccaattgatcgatctgctctttcgttacatttgatcgatctgctctttcgttacatcttctcctaccatggctttcctcgtcttctcctaccatggctttcctcgtaccatcacctctgatcgtgacgttcg

SEQ ID NO: 12 - DNA, 306 bp, Artificial Sequence
  Synthetic DNA
ttgatcgatctgctctttcgttacatcttctcctaccatggctttcctcgtcttctcctaccatggctttcctcgtaccatcacctctgatcgtgacgttcgaccatcacctctgatcgtgacgttcgtatgactgcggataaataccaggaatatgactgcggataaataccaggaactcaccaaacgtctgggtatcaaatcctcaccaaacgtctgggtatcaaatctaccatgtctagcgcaaatcatccgtaccatgtctagcgcaaatcatccgcaaactgatggtcaatccgaacgtac

SEQ ID NO: 13 - DNA, 306 bp, Artificial Sequence
  Synthetic DNA
ctcaccaaacgtctgggtatcaaatctaccatgtctagcgcaaatcatccgtaccatgtctagcgcaaatcatccgcaaactgatggtcaatccgaacgtaccaaactgatggtcaatccgaacgtactatccaaaccctgaatcgtcttctgtatccaaaccctgaatcgtcttctgagggcgtatgctagcaccaacatccaagggcgtatgctagcaccaacatccagaattggcacgtgtacctcccgcaagaattggcacgtgtacctcccgcaaattgaattcgtgtacaattccactcc

SEQ ID NO: 14 - DNA, 306 bp, Artificial Sequence
  Synthetic DNA
agggcgtatgctagcaccaacatccagaattggcacgtgtacctcccgcaagaattggcacgtgtacctcccgcaaattgaattcgtgtacaattccactccattgaattcgtgtacaattccactccgactcgcactctgggtaaatcaccagactcgcactctgggtaaatcaccattcgaaatagatctgggttatctgccttcgaaatagatctgggttatctgcctaatacacccgctatcaaatccgattaatacacccgctatcaaatccgatgatgaagttaacgcgaggagcttcac

SEQ ID NO: 15 - DNA, 306 bp, Artificial Sequence
  Synthetic DNA
ttcgaaatagatctgggttatctgcctaatacacccgctatcaaatccgattaatacacccgctatcaaatccgatgatgaagttaacgcgaggagcttcacgatgaagttaacgcgaggagcttcactgctgtagagctggctaaacatctgtgctgtagagctggctaaacatctgaaagccctgaccatccagaccaaagaaaagccctgaccatccagaccaaagaacagcttgaacatgctcagattgaaacagcttgaacatgctcagattgaaatggaaaccaacaacaaccaacgtcg

SEQ ID NO: 16 - DNA, 306 bp, Artificial Sequence
  Synthetic DNA
aaagccctgaccatccagaccaaagaacagcttgaacatgctcagattgaaacagcttgaacatgctcagattgaaatggaaaccaacaacaaccaacgtcgatggaaaccaacaacaaccaacgtcgtaaacctctgctgttgaacatcggttaaacctctgctgttgaacatcggtgatcatgttctggttcatcgtgatgcgatcatgttctggttcatcgtgatgcatacttcaaaaagggtgcctacatgatacttcaaaaagggtgcctacatgaaagttcagcagatttatgttggccc

SEQ ID NO: 17 - DNA, 308 bp, Artificial Sequence
  Synthetic DNA
gatcatgttctggttcatcgtgatgcatacttcaaaaagggtgcctacatgatacttcaaaaagggtgcctacatgaaagttcagcagatttatgttggcccaaagttcagcagatttatgttggcccttttcgagtggtcaagaagatcaatgttttcgagtggtcaagaagatcaatgacaacgcatatgaactggacctgaacacaacgcatatgaactggacctgaactcccataaaaagaagcatcgtgtgatcccataaaaagaagcatcgtgtgatcaacgtgcaattcctgaaaaagttt

SEQ ID NO: 18 - DNA, 308 bp, Artificial Sequence
  Synthetic DNA
acaacgcatatgaactggacctgaactcccataaaaagaagcatcgtgtgatcccataaaaagaagcatcgtgtgatcaacgtgcaattcctgaaaaagttttcaacgtgcaattcctgaaaaagtttgtttaccgtcctgatgcgtatcctaagtttaccgtcctgatgcgtatcctaaaaacaaaccgatctcttctaccgagcaaacaaaccgatctcttctaccgagcgaatcaaacgcgcccatgaagttacgaatcaaacgcgcccatgaagttacggctctgataggtattgatactaccc

SEQ ID NO: 19 - DNA, 308 bp, Artificial Sequence
  Synthetic DNA
aaacaaaccgatctcttctaccgagcgaatcaaacgcgcccatgaagttacgaatcaaacgcgcccatgaagttacggctctgataggtattgatactacccggctctgataggtattgatactacccacaaaacctacctgtgtcacatgcaaacaaaacctacctgtgtcacatgcaagacgttgacccgactctgtctgttgagacgttgacccgactctgtctgttgaatactctgaagccgaattctgtcagatactctgaagccgaattctgtcagattccagagcgtacccgtcgttctat

SEQ ID NO: 20 - DNA, 308 bp, Artificial Sequence
  Synthetic DNA
gacgttgacccgactctgtctgttgaatactctgaagccgaattctgtcagatactctgaagccgaattctgtcagattccagagcgtacccgtcgttctatattccagagcgtacccgtcgttctatcctggctaattttcgtcaactgtacgcctggctaattttcgtcaactgtacgagactcaggacaacccagaacgtgagagactcaggacaacccagaacgtgaggaagacgtcgtgagccaaaacgaaagaagacgtcgtgagccaaaacgaaatctgccagtacgataacacctctccg

SEQ ID NO: 21 - DNA, 51 bp, Artificial Sequence
  Synthetic DNA
atgaccatcactccagaaacctctcgtccaatcgataccgaaagctggaaa

SEQ ID NO: 22 - DNA, 51 bp, Artificial Sequence
  Synthetic DNA
cacagtggatcagacttgtagtaagatttccagctttcggtatcgattgga

SEQ ID NO: 23 - DNA, 51 bp, Artificial Sequence
  Synthetic DNA
tcttactacaagtctgatccactgtgttccgctgtcctcatccatatgaaa

SEQ ID NO: 24 - DNA, 51 bp, Artificial Sequence
  Synthetic DNA
ggagtaacgttatgctgggtcagttctttcatatggatgaggacagcggaa

SEQ ID NO: 25 - DNA, 51 bp, Artificial Sequence
  Synthetic DNA
gaactgacccagcataacgttactccagaggacatgtctgcatttcgtagc

SEQ ID NO: 26 - DNA, 51 bp, Artificial Sequence
  Synthetic DNA
tcagacagctcgagttttttctggtagctacgaaatgcagacatgtcctct

SEQ ID NO: 27 - DNA, 51 bp, Artificial Sequence; identical to SEQ ID NO: 25
  Synthetic DNA

SEQ ID NO: 28 - DNA, 51 bp, Artificial Sequence; identical to SEQ ID NO: 26
  Synthetic DNA

SEQ ID NO: 29 - DNA, 51 bp, Artificial Sequence
  Synthetic DNA
taccagaaaaaactcgagctgtctgaaacctttcgcaaaaactactccctg

SEQ ID NO: 30 - DNA, 51 bp, Artificial Sequence
  Synthetic DNA
tcttggtagtagatcatttcgtcctccagggagtagtttttgcgaaaggtt

SEQ ID NO: 31 - DNA, 51 bp, Artificial Sequence
  Synthetic DNA
gaggacgaaatgatctactaccaagaccgtctcgtggttccgatcaagcaa

SEQ ID NO: 32 - DNA, 51 bp, Artificial Sequence
  Synthetic DNA
tggtacagacgcataaccgcattttgttgcttgatcggaaccacgagacgg

SEQ ID NO: 33 - DNA, 51 bp, Artificial Sequence; identical to SEQ ID NO: 31
  Synthetic DNA

SEQ ID NO: 34 - DNA, 51 bp, Artificial Sequence; identical to SEQ ID NO: 32
  Synthetic DNA

SEQ ID NO: 35 - DNA, 51 bp, Artificial Sequence
  Synthetic DNA
caaaatgcggttatgcgtctgtaccatgatcatactctgttcggtggtcat

SEQ ID NO: 36 - DNA, 51 bp, Artificial Sequence
  Synthetic DNA
ttagccagagtaacagtaacaccgaaatgaccaccgaacagagtatgatca

SEQ ID NO: 37 - DNA, 51 bp, Artificial Sequence
  Synthetic DNA
ttcggtgttactgttactctggctaaaatctctccgatctactattggccg

SEQ ID NO: 38 - DNA, 51 bp, Artificial Sequence
  Synthetic DNA
tactggatgatagagtgctggagtttcggccaatagtagatcggagagatt

SEQ ID NO: 39 - DNA, 51 bp, Artificial Sequence; identical to SEQ ID NO: 37
  Synthetic DNA

SEQ ID NO: 40 - DNA, 51 bp, Artificial Sequence; identical to SEQ ID NO: 38
  Synthetic DNA

SEQ ID NO: 41 - DNA, 51 bp, Artificial Sequence
  Synthetic DNA
aaactccagcactctatcatccagtacatccgtacctgcgttcagtgtcag

SEQ ID NO: 42 - DNA, 51 bp, Artificial Sequence
  Synthetic DNA
aggcgaggacgatgagatttgatcagctgacactgaacgcaggtacggatg

SEQ ID NO: 43 - DNA, 51 bp, Artificial Sequence
  Synthetic DNA
ctgatcaaatctcatcgtcctcgcctccacggtctcctccagcctctcccg

SEQ ID NO: 44 - DNA, 51 bp, Artificial Sequence
  Synthetic DNA
atgtcaagccaacgaccctcagcgatcgggagaggctggaggagaccgtgg

SEQ ID NO: 45 - DNA, 51 bp, Artificial Sequence; identical to SEQ ID NO: 43
  Synthetic DNA

SEQ ID NO: 46 - DNA, 51 bp, Artificial Sequence; identical to SEQ ID NO: 44
  Synthetic DNA

SEQ ID NO: 47 - DNA, 51 bp, Artificial Sequence
  Synthetic DNA
atcgctgagggtcgttggcttgacattagcatggacttcgtgactggtttg

SEQ ID NO: 48 - DNA, 51 bp, Artificial Sequence
  Synthetic DNA
atgttaagattattagaggtcggcggcaaaccagtcacgaagtccatgcta

SEQ ID NO: 49 - DNA, 51 bp, Artificial Sequence
  Synthetic DNA
ccgccgacctctaataatcttaacatgatcctggttgtagtggaccgattc

SEQ ID NO: 50 - DNA, 51 bp, Artificial Sequence
  Synthetic DNA
gtcgcgatgaagtgagcacgcttagagaatcggtccactacaaccaggatc

SEQ ID NO: 51 - DNA, 51 bp, Artificial Sequence; identical to SEQ ID NO: 49
  Synthetic DNA

SEQ ID NO: 52 - DNA, 51 bp, Artificial Sequence; identical to SEQ ID NO: 50
  Synthetic DNA

SEQ ID NO: 53 - DNA, 51 bp, Artificial Sequence
  Synthetic DNA
tctaagcgtgctcacttcatcgcgactcgtaaaaccctggatgcaacccaa

SEQ ID NO: 54 - DNA, 51 bp, Artificial Sequence
  Synthetic DNA
atgtaacgaaagagcagatcgatcaattgggttgcatccagggttttacga

SEQ ID NO: 55 - DNA, 51 bp, Artificial Sequence
  Synthetic DNA
ttgatcgatctgctctttcgttacatcttctcctaccatggctttcctcgt

SEQ ID NO: 56 - DNA, 51 bp, Artificial Sequence
  Synthetic DNA
cgaacgtcacgatcagaggtgatggtacgaggaaagccatggtaggagaag

SEQ ID NO: 57 - DNA, 51 bp, Artificial Sequence; identical to SEQ ID NO: 55
  Synthetic DNA

SEQ ID NO: 58 - DNA, 51 bp, Artificial Sequence; identical to SEQ ID NO: 56
  Synthetic DNA

SEQ ID NO: 59 - DNA, 51 bp, Artificial Sequence
  Synthetic DNA
accatcacctctgatcgtgacgttcgtatgactgcggataaataccaggaa

SEQ ID NO: 60 - DNA, 51 bp, Artificial Sequence
  Synthetic DNA
gatttgatacccagacgtttggtgagttcctggtatttatccgcagtcata

SEQ ID NO: 61 - DNA, 51 bp, Artificial Sequence
  Synthetic DNA
ctcaccaaacgtctgggtatcaaatctaccatgtctagcgcaaatcatccg

SEQ ID NO: 62 - DNA, 51 bp, Artificial Sequence
  Synthetic DNA
gtacgttcggattgaccatcagtttgcggatgatttgcgctagacatggta

SEQ ID NO: 63 - DNA, 51 bp, Artificial Sequence; identical to SEQ ID NO: 61
  Synthetic DNA

SEQ ID NO: 64 - DNA, 51 bp, Artificial Sequence; identical to SEQ ID NO: 62
  Synthetic DNA

SEQ ID NO: 65 - DNA, 51 bp, Artificial Sequence
  Synthetic DNA
caaactgatggtcaatccgaacgtactatccaaaccctgaatcgtcttctg

SEQ ID NO: 66 - DNA, 51 bp, Artificial Sequence
  Synthetic DNA
tggatgttggtgctagcatacgccctcagaagacgattcagggtttggata

SEQ ID NO: 67 - DNA, 51 bp, Artificial Sequence
  Synthetic DNA
agggcgtatgctagcaccaacatccagaattggcacgtgtacctcccgcaa

SEQ ID NO: 68 - DNA, 51 bp, Artificial Sequence
  Synthetic DNA
ggagtggaattgtacacgaattcaatttgcgggaggtacacgtgccaattc

SEQ ID NO: 69 - DNA, 51 bp, Artificial Sequence; identical to SEQ ID NO: 67
  Synthetic DNA

SEQ ID NO: 70 - DNA, 51 bp, Artificial Sequence; identical to SEQ ID NO: 68
  Synthetic DNA

SEQ ID NO: 71 - DNA, 51 bp, Artificial Sequence
  Synthetic DNA
attgaattcgtgtacaattccactccgactcgcactctgggtaaatcacca

SEQ ID NO: 72 - DNA, 51 bp, Artificial Sequence
  Synthetic DNA
ggcagataacccagatctatttcgaatggtgatttacccagagtgcgagtc

SEQ ID NO: 73 - DNA, 51 bp, Artificial Sequence
  Synthetic DNA
ttcgaaatagatctgggttatctgcctaatacacccgctatcaaatccgat

SEQ ID NO: 74 - DNA, 51 bp, Artificial Sequence
  Synthetic DNA
gtgaagctcctcgcgttaacttcatcatcggatttgatagcgggtgtatta

SEQ ID NO: 75 - DNA, 51 bp, Artificial Sequence; identical to SEQ ID NO: 73
  Synthetic DNA

SEQ ID NO: 76 - DNA, 51 bp, Artificial Sequence; identical to SEQ ID NO: 74
  Synthetic DNA

SEQ ID NO: 77 - DNA, 51 bp, Artificial Sequence
  Synthetic DNA
gatgaagttaacgcgaggagcttcactgctgtagagctggctaaacatctg

SEQ ID NO: 78 - DNA, 51 bp, Artificial Sequence
  Synthetic DNA
tctttggtctggatggtcagggctttcagatgtttagccagctctacagca

SEQ ID NO: 79 - DNA, 51 bp, Artificial Sequence
  Synthetic DNA
aaagccctgaccatccagaccaaagaacagcttgaacatgctcagattgaa

SEQ ID NO: 80 - DNA, 51 bp, Artificial Sequence
  Synthetic DNA
cgacgttggttgttgttggtttccatttcaatctgagcatgttcaagctgt

SEQ ID NO: 81 - DNA, 51 bp, Artificial Sequence; identical to SEQ ID NO: 79
  Synthetic DNA

SEQ ID NO: 82 - DNA, 51 bp, Artificial Sequence; identical to SEQ ID NO: 80
  Synthetic DNA

SEQ ID NO: 83 - DNA, 51 bp, Artificial Sequence
  Synthetic DNA
atggaaaccaacaacaaccaacgtcgtaaacctctgctgttgaacatcggt

SEQ ID NO: 84 - DNA, 51 bp, Artificial Sequence
  Synthetic DNA
gcatcacgatgaaccagaacatgatcaccgatgttcaacagcagaggttta

SEQ ID NO: 85 - DNA, 51 bp, Artificial Sequence
  Synthetic DNA
gatcatgttctggttcatcgtgatgcatacttcaaaaagggtgcctacatg

SEQ ID NO: 86 - DNA, 51 bp, Artificial Sequence
  Synthetic DNA
gggccaacataaatctgctgaactttcatgtaggcaccctttttgaagtat

SEQ ID NO: 87 - DNA, 51 bp, Artificial Sequence; identical to SEQ ID NO: 85
  Synthetic DNA

SEQ ID NO: 88 - DNA, 51 bp, Artificial Sequence; identical to SEQ ID NO: 86
  Synthetic DNA

SEQ ID NO: 89 - DNA, 52 bp, Artificial Sequence
  Synthetic DNA
aaagttcagcagatttatgttggcccttttcgagtggtcaagaagatcaatg

SEQ ID NO: 90 - DNA, 52 bp, Artificial Sequence
  Synthetic DNA
gttcaggtccagttcatatgcgttgtcattgatcttcttgaccactcgaaaa

SEQ ID NO: 91 - DNA, 51 bp, Artificial Sequence
  Synthetic DNA
acaacgcatatgaactggacctgaactcccataaaaagaagcatcgtgtga

SEQ ID NO: 92 - DNA, 51 bp, Artificial Sequence
  Synthetic DNA
aaactttttcaggaattgcacgttgatcacacgatgcttctttttatggga

SEQ ID NO: 93 - DNA, 51 bp, Artificial Sequence; identical to SEQ ID NO: 91
  Synthetic DNA

SEQ ID NO: 94 - DNA, 51 bp, Artificial Sequence; identical to SEQ ID NO: 92
  Synthetic DNA

SEQ ID NO: 95 - DNA, 52 bp, Artificial Sequence
  Synthetic DNA
tcaacgtgcaattcctgaaaaagtttgtttaccgtcctgatgcgtatcctaa

SEQ ID NO: 96 - DNA, 52 bp, Artificial Sequence
  Synthetic DNA
gctcggtagaagagatcggtttgtttttaggatacgcatcaggacggtaaac

SEQ ID NO: 97 - DNA, 51 bp, Artificial Sequence
  Synthetic DNA
aaacaaaccgatctcttctaccgagcgaatcaaacgcgcccatgaagttac

SEQ ID NO: 98 - DNA, 51 bp, Artificial Sequence
  Synthetic DNA
gggtagtatcaatacctatcagagccgtaacttcatgggcgcgtttgattc

SEQ ID NO: 99 - DNA, 51 bp, Artificial Sequence; identical to SEQ ID NO: 97
  Synthetic DNA

SEQ ID NO: 100 - DNA, 51 bp, Artificial Sequence; identical to SEQ ID NO: 98
  Synthetic DNA

SEQ ID NO: 101 - DNA, 52 bp, Artificial Sequence
  Synthetic DNA
ggctctgataggtattgatactacccacaaaacctacctgtgtcacatgcaa

SEQ ID NO: 102 - DNA, 52 bp, Artificial Sequence
  Synthetic DNA
tcaacagacagagtcgggtcaacgtcttgcatgtgacacaggtaggttttgt

SEQ ID NO: 103 - DNA, 51 bp, Artificial Sequence
  Synthetic DNA
gacgttgacccgactctgtctgttgaatactctgaagccgaattctgtcag

SEQ ID NO: 104 - DNA, 51 bp, Artificial Sequence
  Synthetic DNA
atagaacgacgggtacgctctggaatctgacagaattcggcttcagagtat

SEQ ID NO: 105 - DNA, 51 bp, Artificial Sequence; identical to SEQ ID NO: 103
  Synthetic DNA

SEQ ID NO: 106 - DNA, 51 bp, Artificial Sequence; identical to SEQ ID NO: 104
  Synthetic DNA

SEQ ID NO: 107 - DNA, 52 bp, Artificial Sequence
  Synthetic DNA
attccagagcgtacccgtcgttctatcctggctaattttcgtcaactgtacg

SEQ ID NO: 108 - DNA, 52 bp, Artificial Sequence
  Synthetic DNA
ctcacgttctgggttgtcctgagtctcgtacagttgacgaaaattagccagg

SEQ ID NO: 109 - DNA, 51 bp, Artificial Sequence
  Synthetic DNA
agactcaggacaacccagaacgtgaggaagacgtcgtgagccaaaacgaaa

SEQ ID NO: 110 - DNA, 51 bp, Artificial Sequence
  Synthetic DNA
cggagaggtgttatcgtactggcagatttcgttttggctcacgacgtcttc

(19) United States
(12) Patent Application Publication
(10) Pub. No.: US 2007/0009928 A1
(43) Pub. Date: Jan. 11, 2007

(54) Gene synthesis using pooled DNA

(76) Inventors: Richard H. Lathrop, Irvine, CA (US); She-Pin Hung, Aliso Viejo, CA (US); Richard Colman, Laguna Hills, CA (US); G. Wesley Hatfield, Corona del Mar, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/393,043

(22) Filed: Mar. 30, 2006

Related U.S. Application Data
(60) Provisional application No. 60/667,108, filed on Mar. 31, 2005.

(51) Int. Cl. C12Q 1/68; G06F 19/00; C12P 19/34

(52) U.S. Cl. 435/6; 435/91.2; 702/20

(57) ABSTRACT

A method and system for synthesizing one or more pieces of DNA with desired sequences using pooled DNA, the method comprising a hierarchical division phase and a hierarchical assembly phase. In the division phase, the sequences of one or more pieces of DNA with desired nucleic acid sequences are recursively: divided into partially overlapping resulting pieces of DNA, and the resulting pieces of DNA assigned to a plurality of pools except after the after the final division step, wherein overlapping, adjacent resulting pieces of DNA are assigned to different pools. In the assembly phase, pools of oligonucleotides are obtained corresponding to the pools of the resulting pieces of DNA, and one or more pieces of DNA with desired sequences are assembled by overlap extension in the reverse order of the hierarchical division. Embodiments of the method combine the advantages of hierarchical assembly with the advantages of pooled oligonucleotides.

### CROSS-REFERENCE TO RELATED APPLICATIONS

This application claims the benefit of U.S. application No. 60/667,108, filed Mar. 31, 2005, the disclosure of which is incorporated by reference.

### BACKGROUND OF THE INVENTION

1. Field of the Invention

This application is generally related to the synthesis of DNA molecules, and more particularly, to the synthesis of a synthetic gene or other DNA sequence.

2. Description of the Related Art

Proteins are an important class of biological molecules that have a wide range of valuable medical, pharmaceutical, industrial, and biological applications. A gene encodes the information necessary to produce a protein according to the genetic code using three nucleotides (one codon or set of codons) for each amino acid in the protein. An expression vector contains DNA sequences that allow transcription of the gene into mRNA for translation into a protein.

It is often desirable to obtain a synthetic DNA that encodes the protein of interest. Typically, DNA can be synthesized accurately by chemical coupling only in short pieces of about 50 to 80 nucleotides or fewer. Chemical synthesis of substantially longer pieces is problematic because of cumulative error probability in the synthesis process. Genes are typically appreciably longer than 50 to 80 nucleotides, usually by hundreds or thousands of nucleotides. Consequently, direct synthesis is not a convenient method for producing large genes.

A gene that does not contain introns can often be synthesized by PCR directly from genomic DNA. This method is feasible for genes of bacteria, lower eukaryotes, and many viruses. Nearly all genes of higher organisms contain introns, however. A related alternative is to PCR the gene from a full-length cDNA clone. Isolating and characterizing a full-length clone is often time consuming, and tedious, and full-length cDNA clones are available for only a very small fraction of the genes of many higher organisms of interest.

06 In some strategies, synthetic genes are assembled from a large number of short partially overlapping DNA segments, called oligonucleotides. Adjacent overlapping oligonucleotides comprise sequences from opposite (Watson and Crick) strands of the desired gene and have complementary overlapping ends. These segments are allowed to anneal and then assembled into longer double-stranded DNA, for example, by ligation and/or polymerase extension reactions, either alone or in combination.

Current processes are referred to variously as “assembly PCR,” “splicing by overlap extension,” “polymerase chain assembly,” “recursive PCR,” and others. See, for example, W. P. C. Stemmer et al. “Single-step assembly of a gene and entire plasmid from large numbers of oligodeoxyribonucleotides” Gene, 1995, 164, 49-53 and D. E. Casimiro et al. “PCR-based gene synthesis and protein NMR spectroscopy” Structure, 1997, 5, 1407-1412. An method for automated design of the oligonucleotides for gene synthesis by this approach has recently been described in D. M. Hoover & J. Lubkowski “DNA Works: an automated method for designing oligonucleotides for PCR-based gene synthesis” Nucleic Acids Res., 2002, 30:10 e43.

In these methods, the DNA fragments, segments, and/or oligonucleotides often assemble incorrectly due to incorrect annealing, for example, mis-priming and/or cross-hybridization, of the complementary, overlapping ends. Such incorrect assembly results in a mixture of products of varying lengths, containing the correct product mixed with a large number of incorrect products. When visualized on an electrophoresis gel, for example, the resulting mixture provides a smeared or diffuse band, as seen for example in FIG. 2 of Hoover and Lubowski (2002), in FIG. 3 of Smith et al. (2003), or in FIG. 6 of Richmond et al. (2004).

The probability that DNA segments will assemble incorrectly increases as the square of the number of segments because every segment potentially can mis-prime or cross-hybridize with every other segment. To address this problem, synthetic genes are often assembled hierarchically (hierarchical assembly), for example. At the first step, small groups of contiguous overlapping synthetic oligonucleotides are assembled into intermediate DNA fragments. At the next step, small groups of contiguous overlapping intermediate DNA fragments are assembled into larger intermediate DNA fragments. This process is continued until the complete gene is produced. The advantage is that assembly errors are reduced and the yield of correct product is increased. The disadvantage is that more assembly steps are required, so the process is more expensive and time-consuming.

Recently, two reports have demonstrated DNA assembly from pools of oligonucleotides from DNA chips (Richmond et al. 2004, Tian et al. 2004). Both synthesized multiple oligonucleotides in parallel on a DNA chip and released them from the substrate. Both fabricated the oligos with constant PCR leaders and trailers so that the oligos could be amplified en masse in a single PCR reaction using constant primers. The leaders and trailers were removed from the oligos after amplification using restriction enzymes. Both approaches then used the digested oligos, minus primers, in an assembly PCR reaction to create longer DNA fragments.

Richmond, K. E., et al., “Amplification and assembly of chip-eluted DNA (AACED): a method for high-throughput gene synthesis” Nucleic Acids Research, 2004, 32(17):5011-5018 reports that synthesis of a 60 base pair DNA construct from the assembly PCR reaction as proof of concept that the oligonucleotides were biologically active. This was the first report demonstrating the use of released (eluted) oligonucleotides from DNA chips in biological applications such as assembly PCR. Because their synthesized DNA construct was so short, they were able to neglect the challenges of incorrect assembly due to mis-priming and of removing synthetic point defects.

Tian, J., et al., “Accurate multiplex gene synthesis from programmable DNA microchips” Nature, 2004, 432:1050-1054 reports the synthesis of all 21 genes that encode the proteins of the E. coli 30S ribosomal subunit in a first demonstration of multiplexed biological gene synthesis from DNA chips. A special-purpose computer-aided design software (CAD-PAM, cited as “manuscript in preparation” ) addressed the problem of incorrect assembly due to mis-priming. Synthetic point defects were removed by a two-step hybridization filter in which construction oligonucleotides were hybridized sequentially to two pools of bead-immobilized selection oligonucleotides, which were also synthesized and released from DNA chips. Each selection pool was designed to hybridize to half of each construction oligonucleotide, and collectively they spanned the entire DNA construction. Hybridization thermodynamics favored correct oligonucleotides.

Zhou, X., et al., “Microfluidic PicoArray synthesis of oligodeoxynucleotides and simultaneous assembling of multiple DNA sequences” Nucl. Acids Res., 2004, 32(18):5409-5417, 2004 describes a microfluidic chamber which was used for the multiplexed synthesis of DNA oligonucleotides, which were later used to synthesize a small gene.

Other related work includes: Engels, J. W., “Gene synthesis on microchips” Angew. Chem. Int. Ed. 2005, 44(44): 7166-7169; Gao, X., et al., “Thermodynamically balanced inside-out (TBIO) PCR-based gene synthesis” Nucl. Acids Res., 2003, 31(22):e142; Jayaraj, S., et al., “GeMS: an advanced software package for designing synthetic genes” Nucl. Acids Res., 2005, 33(9):3011-3016; Kodumal, et al., “Total synthesis of long DNA sequences” Proc. Natl. Acad. Sciences, USA, November 2004, 101(44):15573-15578; Rouillard, J.-M., et al., “Gene2Oligo: oligonucleotide design for in vitro gene synthesis” Nucl. Acids Res., 2004, 32:W176-180; Rydzanicz, R., et al., “Assembly PCR oligo maker” Nucl. Acids Res., 2005, 33:W521-525; Saboulard D, Dugas V, Jaber M, et al., “High-throughput site-directed mutagenesis using oligonucleotides synthesized on DNA chips” Biotechniques, September 2005, 39(3):363-368; Smith, H. O., et al., Proc. Natl. Acad. Sciences, USA, December 2003, 100(26):15440-15445; Xiong, A.-S., et al., “A simple, rapid, high-fidelity and cost-effective PCR-based two-step DNA synthesis method for long gene sequences” Nucl. Acids Res., 2004, 32(12):e98; and Young, L., Dong, Q., “Two-step total gene synthesis method” Nucl. Acids Res., 2004, 32(7):e59.

Fueled by growing demand from academic researchers, industry, and government, there is a large need for improvements in gene synthesis speed, cost, accuracy, and scalability.

### SUMMARY OF THE INVENTION

A method and system for synthesizing one or more pieces of DNA with desired sequences using pooled DNA, the method comprising a hierarchical division phase and a hierarchical assembly phase. In the division phase, the sequences of one or more pieces of DNA with desired nucleic acid sequences are recursively: divided into partially overlapping resulting pieces of DNA, and the resulting pieces of DNA assigned to a plurality of pools except after the after the final division step, wherein overlapping, adjacent resulting pieces of DNA are assigned to different pools. In the assembly phase, pools of oligonucleotides are obtained corresponding to the pools of the resulting pieces of DNA, and one or more pieces of DNA with desired sequences are assembled by overlap extension in the reverse order of the hierarchical division. Embodiments of the method combine the advantages of hierarchical assembly with the advantages of pooled oligonucleotides.

Some embodiments provide a method for hierarchically synthesizing a piece of DNA with a desired nucleic acid sequence, the method comprising a hierarchical division of a nucleic acid sequence of a piece of DNA with a desired nucleic acid sequence by a method comprising: (i) hierarchically dividing the nucleic acid sequence into a plurality of DNA sequences, wherein adjacent DNA sequences comprise overlapping portions; (ii) optionally, optimizing at least some of the DNA sequences to strengthen correct hybridizations between the overlapping portions of adjacent DNA sequences and to weaken incorrect hybridizations; (iii) assigning, at each hierarchical level of division except a final hierarchical level of division, the DNA sequences into a plurality of pools of DNA sequences, wherein adjacent. DNA sequences with overlapping portions are assigned to different pools; and (iv) recursively repeating steps (i), (ii), and (iii) for each DNA sequence in each pool.

In some embodiments, the piece of DNA with a desired nucleic acid sequence is a member of a pool comprising a plurality of pieces of DNA with desired nucleic acid sequences, and the hierarchical division is simultaneously performed on the nucleic acid sequences of the plurality of pieces of DNA with desired nucleic acid sequences. In some embodiments, in at least one hierarchical level of division, all of the DNA sequences are about the same size.

In some embodiments, the method comprise optimizing within at least one hierarchical level of division, and the optimizing comprises globally optimizing all possible correct and incorrect hybridizations between every DNA sequence in at least one pool. In some embodiments, the method comprise optimizing within at least one hierarchical level of division, and the optimizing comprises calculating a temperature gap between a melting temperature of a lowest correct hybridization and a melting temperature of a highest incorrect hybridization. In some embodiments, the temperature gap is at least about 1° C. In some embodiments, the method comprises optimizing within at least one hierarchical level of division, and optimizing comprises permuting a silent codon substitution. In some embodiments, the silent codon substitution is a substitution according to a codon usage preference for an organism. In some embodiments, the codon usage preference is a codon pair preference. In some embodiments, the organism is E. coli. In some embodiments, the method comprises optimizing within at least one hierarchical level of division, and optimizing comprises taking advantage of a degeneracy in a regulatory region consensus sequence. In some embodiments, the method comprises optimizing within at least one hierarchical level of division, and optimizing comprises adjusting boundary points between adjacent resulting pieces of DNA. In some embodiments, the optimizing in at least one hierarchical level of division comprises direct base assignment.

In some embodiments, at least one of the pools comprises at least some of the DNA sequences resulting from a division of a plurality of next-larger DNA sequence from a next-higher hierarchical level of division. In some embodiments, the pools are maximal pools.

In some embodiments, the method is automated.

Other embodiments provide a method for hierarchically synthesizing a piece of DNA with a desired nucleic acid sequence, the method comprising a hierarchical assembly of a piece of DNA with a desired nucleic acid sequence by a method comprising: (v) obtaining pools of pieces of DNA corresponding to pools of DNA sequences of a final hierarchical division produced according to the disclosed method for hierarchically dividing a nucleic acid sequence of the piece of DNA with a desired nucleic acid sequence; (vi) allowing the pieces of DNA in each pool to self-assemble into DNA constructs corresponding to next-larger pieces of DNA in a next-higher hierarchical level of division; (vii) producing the next-larger pieces of DNA from the DNA constructs; (viii) creating pools of the next-larger pieces of DNA corresponding to the next-higher hierarchical level of the division; and (ix) recursively repeating steps (vi), (vii), and (viii) in the reverse order of the hierarchical division in steps (i), (ii), (iii), and (iv) to synthesize the piece of DNA with a desired nucleic acid sequence.

In some embodiments, the pieces of DNA in step (v) are synthetic oligonucleotides.

In some embodiments, in at least one hierarchical level of assembly, the next-larger pieces of DNA are about the same size.

In some embodiments, in at least one hierarchical level of assembly, producing the next-larger pieces of DNA comprises polymerase overlap extension or ligation. In some embodiments, in at least one hierarchical level of assembly, producing the next-larger pieces of DNA comprises polymerase overlap extension; and the polymerase overlap extension comprises a high-fidelity DNA polymerase reaction.

Some embodiments further comprise in at least one hierarchical level of assembly, at least one of purifying or amplifying the next-larger pieces of DNA after at least one of steps (vii) or (viii). In some embodiments, the purifying comprises at least one of electrophoresis or chromatography. In some embodiments, the purifying comprises treatment with an enzyme. In some embodiments, the enzyme is MutS, T7 endonuclease, or a combination thereof. In some embodiments, the amplifying comprises a polymerase chain reaction.

In some embodiments, at least one of steps (vi), (vii), or (viii) is automated. In some embodiments, at least one of the automated steps is performed microfluidically.

In some embodiments, the piece of DNA with a desired nucleic acid sequence is a member of a pool comprising a plurality of pieces of DNA with desired nucleic acid sequences, and the pools of pieces of DNA in step (v) correspond to pools of DNA sequences of a final hierarchical division produced according to the method of claim 1 performed on the nucleic acid sequences of the plurality of pieces of DNA with desired nucleic acid sequences. In some embodiments, the product of the final hierarchical assembly comprises a pool of pieces of DNA with desired sequences.

Some embodiments further comprise isolating at least one piece of DNA with a desired sequence after the last hierarchical assembly step. In some embodiments, the isolating comprises a polymerase chain reaction. Some embodiments further comprise selecting a piece of DNA with a desired sequence after the last hierarchical assembly step. In some embodiments, the selection comprises cloning the piece of DNA with a desired sequence into a frameshift vector. In some embodiments, the frameshift vector comprises SEQ. ID. NO.: 1 or SEQ. ID. NO.: 2.

Some embodiments further comprise producing pools of oligonucleotides corresponding to the pools of DNA sequences of the final hierarchical division produced according to the disclosed method for hierarchically dividing a nucleic acid sequence for a piece of DNA with a desired nucleic acid sequence. In some embodiments, at least one of the pools of oligonucleotides comprises oligonucleotides bound to a solid-state support. In some embodiments, the solid-state support comprises beads, an array, or combinations thereof.

Some embodiments provide a system for hierarchically synthesizing a piece of DNA with a desired nucleic acid sequence, the system comprising a plurality of pools of oligonucleotides corresponding to pools of DNA sequences of a final hierarchical division produced according to the disclosed method for hierarchically dividing a nucleic acid sequence for a piece of DNA with a desired nucleic acid sequence performed on a nucleic acid sequence of a piece of DNA with a desired nucleic acid sequence.

In some embodiments, at least one pool of oligonucleotides is disposed in a tube or in a well. In some embodiments, at least one pool of oligonucleotides is bound to a solid-state support. In some embodiments, the solid-state support comprises beads, an array, or combinations thereof. In some embodiments, the piece of DNA with a desired nucleic acid sequence is a member of a pool comprising a plurality of pieces of DNA with desired nucleic acid sequences, and the plurality of pools of oligonucleotides correspond to pools of DNA sequences of a final hierarchical division produced according to the disclosed method for hierarchically dividing a nucleic acid sequence for a piece of DNA with a desired nucleic acid sequence performed on the nucleic acid sequences of the plurality of pieces of DNA with desired nucleic acid sequences. Some embodiments further comprise polymerase chain reaction primers suitable for isolating at least one piece of DNA with a desired nucleic acid sequence. Some embodiments further comprise a frameshift vector. Some embodiments further comprise instructions for synthesizing a piece of DNA with a desired nucleic acid sequence from the plurality of pools of oligonucleotides.

Some embodiments provide a system for hierarchically synthesizing a piece of DNA with a desired nucleic acid sequence, the system comprising machine readable media comprising machine readable instructions, which when executed, perform the disclosed method for hierarchically dividing a nucleic acid sequence for a piece of DNA with a desired nucleic acid sequence. Some embodiments further comprise a data processing unit operatively coupled to the machine readable media, wherein the data processing unit is operable to execute the machine readable instructions.

Some embodiments provide a plurality of pools of DNA sequences corresponding to a plurality of pools of DNA sequences of a final hierarchical division produced according to the disclosed method for hierarchically dividing a nucleic acid sequence for a piece of DNA with a desired nucleic acid sequence performed on a nucleic acid sequence of a piece of DNA with a desired nucleic acid sequence. In some embodiments, the pools of DNA sequences are provided in a fixed medium. In some embodiments, the piece of DNA with a desired nucleic acid sequence is a member of a pool comprising a plurality of pieces of DNA with desired nucleic acid sequences, and the plurality of pools of DNA sequences correspond to pools of DNA sequences of a final hierarchical division produced by the method of claim 1 performed on the nucleic acid sequences of the plurality of pieces of DNA with desired nucleic acid sequences.

### BRIEF DESCRIPTION OF THE DRAWINGS

FIG. 1 is a flowchart illustrating a method for synthesizing one or more synthetic genes using pooled DNA.

FIGS. 2A-2F schematically illustrate an embodiment of a three-level hierarchical division and assembly of a synthetic gene.

FIG. 3 schematically illustrates embodiments of different methods for purifying and/or amplifying next-larger pieces of DNA.

FIGS. 4A-C schematically illustrate embodiments of microfluidic implementations for purifying and/or amplifying next-larger pieces of DNA.

FIG. 5 schematically illustrates an embodiment of a method for synthesizing synthetic genes from pooled DNA.

FIG. 6 schematically illustrates an embodiment of a method for synthesizing synthetic genes from pooled DNA.

FIG. 7A-7C are agarose gel electrophoresis analyses of intermediates and the final product of the synthesis of yeast Ty3 1N described in the EXAMPLE.

### DETAILED DESCRIPTION OF THE PREFERRED EMBODIMENT

As used herein, the term “DNA” includes both single-stranded and doubled-stranded DNA. The term “piece of DNA” refers to both physical pieces of DNA as well as DNA sequences, according to the context. The term “adjacent” in the context of pieces of DNA, DNA fragments, and oligonucleotides refers to pieces of DNA that at least partially overlap. The term “gene” is used in-its usual sense, as well as to refer to large pieces of DNA of any function and/or sequence, pieces of DNA comprising one or more open reading frames, pieces of DNA comprising one or more opening reading frames and one or more regulatory sequences, and/or substantially complete genomes. The term “intermediate fragment” as used herein refers to pieces of DNA synthesized in the course of the synthesis of the synthetic gene according to the disclosed method. A “desired nucleic acid sequence” includes both a specific nucleotide sequence, as well as a nucleotide sequence that is equivalent under the relevant context. For example, in the context of polypeptide expression, a desired nucleic acid sequence includes a particular sequence that encodes a specific polypeptide, as well as one of the many nucleic acid sequences that encode the specific polypeptide. The disclosures of all references cited herein are incorporated by reference.

Disclosed are a hierarchical method and system for synthesizing one or more DNA sequence using pooled DNA oligonucleotides by assigning overlapping intermediate fragments to different pools, and the DNA sequences synthesized by the method. In some embodiments, the oligonucleotide pools comprise oligonucleotides designed to synthesize a plurality of synthetic genes. In some embodiments, a plurality of synthetic genes is simultaneously synthesized from the pooled oligonucleotides. In other embodiments, a single synthetic gene is selectively synthesized from the pooled oligonucleotides.

Some embodiments of the disclosed method include one or more advantages compared to known methods of gene synthesis from pooled oligonucleotides. For example, the disclosed method allows for simple PCR amplification of each intermediate fragment produced in the initial assembly step because each of these intermediate fragments is comparatively short and the adjacent intermediate fragment(s), which comprise overlapping sequences that would otherwise interfere with the PCR amplification, are synthesized separately. PCR of short fragments is generally easier and more reliable. In succeeding assembly steps after the initial step, there are generally fewer participants in each reaction, with longer overlaps, and fewer connections to-be-made. Accordingly, some embodiments of the method provide simpler, easier, and/or better multiplex gene assembly compared with other methods. Some embodiments of the disclosed method produce, at each hierarchical level, intermediate fragments of the same or about the same length. Accordingly, some embodiments of the method provide simpler, easier, and/or better purification of the intermediate fragments compared with other methods.

Those skilled in the art will immediately comprehend myriad applications to which the disclosed method may be applied, including: (1) creating de novo “designer” proteins; (2) coupling to automated expression and crystallization facilities; (3) building DNA sequences predicted to express novel protein folds for structural proteomics; (4) building other DNA sequences that do not encode proteins, e.g., as RNA structural templates or DNA nanotechnology components; (5) expressing proteins from a different species in a desired expression vector according to its own codon usage preference; and (6) creating a small synthetic genome by specifying its desired protein sequences and regulatory protein binding sites.

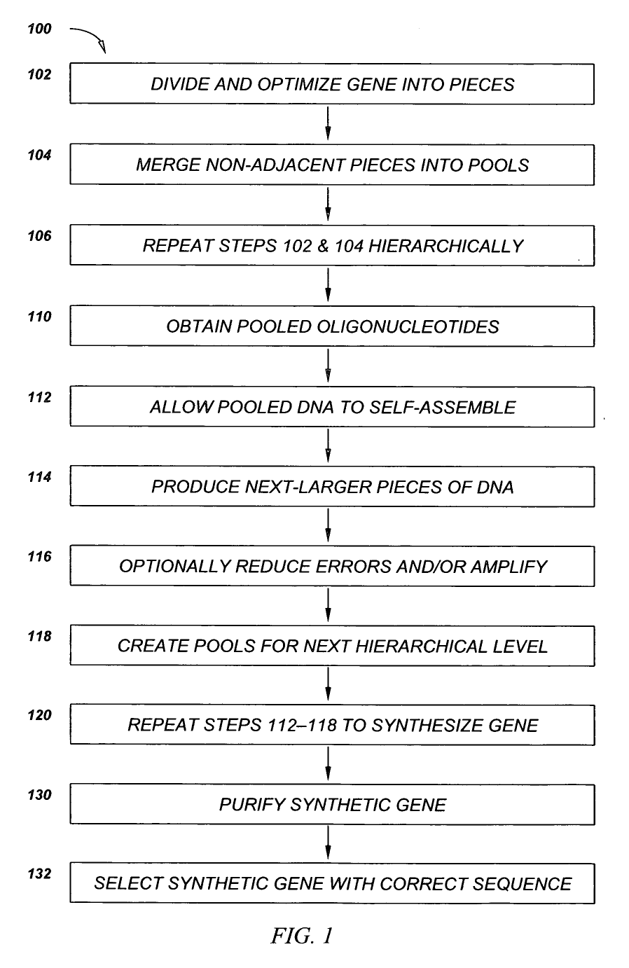

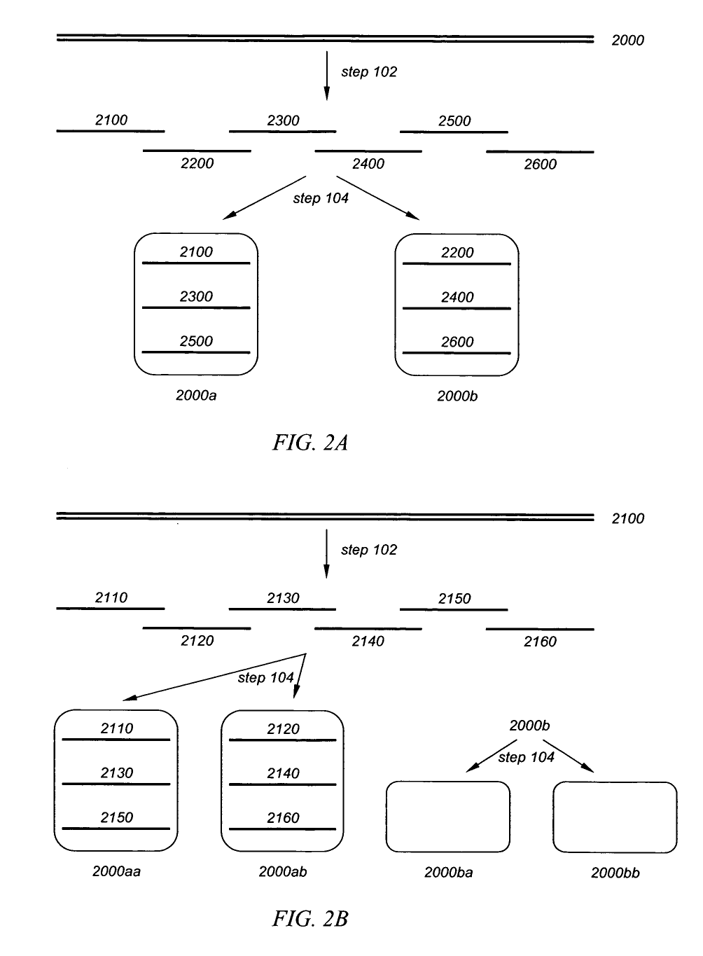

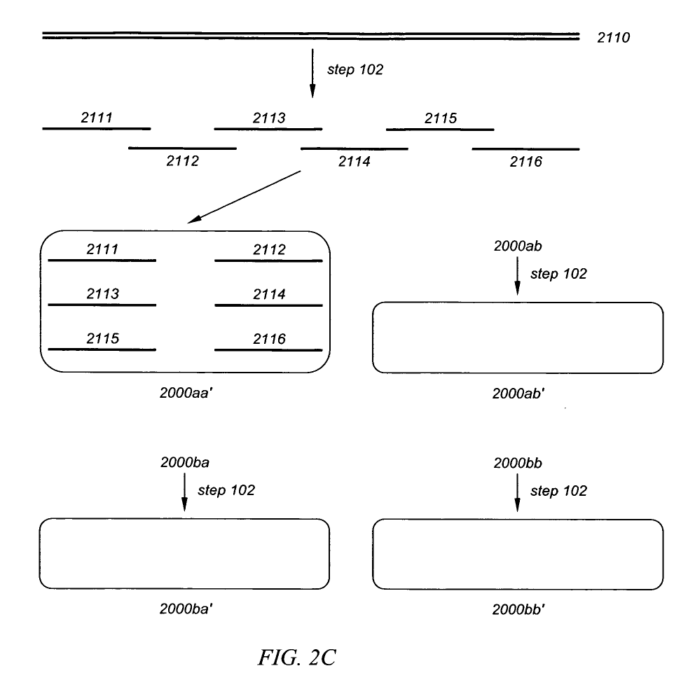

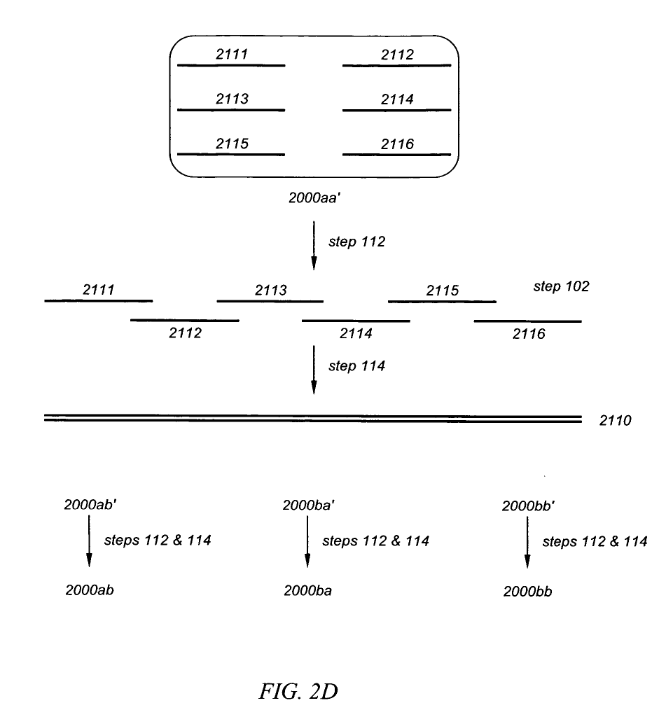

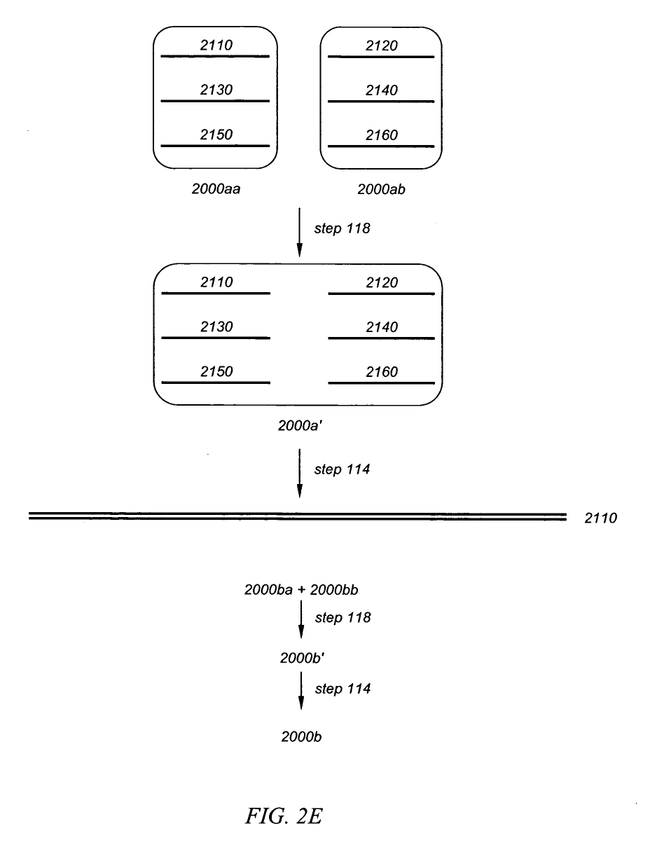

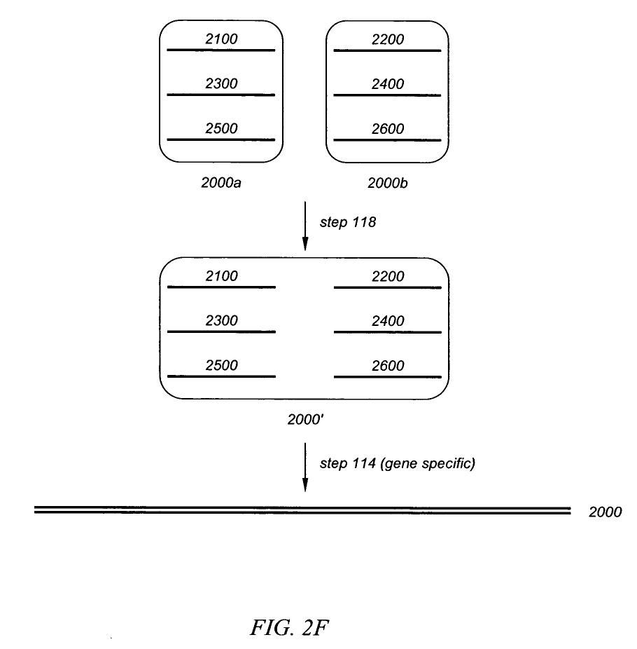

FIG. 1 is a flowchart illustrating an embodiment of a method 100 for synthesizing DNA sequences. The method 100 generally comprises two phases: a division phase comprising steps 102, 104, and 106, and an assembly phase comprising steps 110, 112, 116, 118, 120. The following description of the method 100 references FIGS. 2A-21F, which schematically illustrates the division of a synthetic gene and assembly of the same using a three-level hierarchical method.

In step 102, the desired DNA sequence or sequences are hierarchically divided into a plurality of partially overlapping pieces of DNA or oligonucleotides. In step 104, at each hierarchical level of division except the final division step, the resulting pieces of DNA are assigned to pools of DNA pieces, wherein overlapping adjacent pieces of DNA are assigned to different pools. In step 106, steps 102 and 104 are repeated hierarchically for each pool. In step 110, pools of synthetic DNA are obtained corresponding to the smallest pieces of DNA identified in the final hierarchical division step. In step 112, the pieces of DNA in each pool are allowed to self-assemble into DNA constructs corresponding to the next-larger pieces of DNA in the next-higher hierarchical level of the division. In step 114, the next-larger pieces of DNA are produced from the DNA constructs by polymerase overlap extension and/or by ligation. In optional step 116, errors one or more of the next-larger pieces of DNA are reduced, for example, by purification and/or amplification. In step 118, pools of the next-larger pieces of DNA corresponding to the next-higher hierarchical level of the division are produced. In step 120, assembly steps 112-118 are repeated in the reverse order of the division steps 102-106 to synthesize the synthetic gene(s). In step 130, the synthetic gene(s) are isolated. In step 132, synthetic gene(s) with the desired sequence are selected. Embodiments of steps 102, 110, 112, 118 are described in greater detail in U.S. Patent Publication Nos. 2004/0235035 A1 and 2005/0106590 A1. Embodiments of steps 130 and 132 are described in greater detail in U.S. Patent Publication No.2005/0106590 A1.

In optional step 102, the gene or genes are divided into pieces of DNA designed and optimized to encode their own correct self-assembly by hierarchical assembly, for example, intermediate fragments and/or oligonucleotides. In some preferred embodiments, the division and optimization in step 102 is performed as disclosed in U.S. Patent Publication Nos. 2004/0235035 A1 and 2005/0106590 A1. In embodiments in which a plurality of synthetic genes is synthesized simultaneously, the division and optimization process in step 102 includes all of the synthetic genes. In the optimization, correct hybridizations between adjacent, overlapping pieces of DNA are strengthened, and incorrect hybridizations are weakened. Correct hybridizations are the designed or desired hybridizations between the overlapping portions of adjacent pieces of DNA. Incorrect hybridizations are all other hybridizations, including, for example, hybridizations within a piece of DNA (e.g., hairpins), undesired hybridizations to a non-overlapping portion of an adjacent piece of DNA, and any hybridizations between non-adjacent pieces of DNA. In some preferred embodiments, the optimization is global, that is, all possible hybridizations between all of pieces of DNA are evaluated. In some embodiments, the global optimization is performed between all pieces of DNA in each pool. Pools are discussed in greater detail below.

Briefly, optimization for self-assembly is performed by calculating one or more parameters or measures related to hybridization propensity for all correct and incorrect hybridizations in a pool of DNA pieces, for example, melting temperature, free energy, enthalpy, entropy, or other arithmetic or algebraic combinations of such parameters or measures. In some preferred embodiments, the parameter is a melting temperature. Indeed, the melting temperature itself is one such arithmetic or algebraic combination of such parameters or measures. A melting temperature gap between the correct and incorrect hybridizations is then determined. Preferably, the lowest correct hybridization melting temperature is higher than the highest incorrect hybridization melting temperature. This melting temperature gap is then optimized or increased. In some embodiments, the pieces of DNA are optimized by permuting silent codon substitutions, for example for a portion encoding a polypeptide. In some embodiments, the silent codon substitution is a substitution according to a codon usage preference for an organism, for example, for E. coli or another suitable organism known in the art. In some embodiments, the codon usage preference is a codon pair preference, for example, as disclosed in U.S. Pat. No. 5,082,767. In some embodiments, the pieces of DNA are optimized by taking advantage of the degeneracy in the regulatory region consensus sequence, for example for a regulatory region. In some embodiments, the pieces of DNA are optimized by adjusting boundary points between adjacent pieces of DNA. In some embodiments, the pieces of DNA are optimized by direct base assignment, for example for an intergenic region.

Those skilled in the art will realize that the size of such a melting temperature gap affects the annealing conditions used in the assembly steps discussed below. Because the stringency of the annealing conditions is adjustable to provide annealing with any desired level of ideality, there is no theoretical minimum value for the temperature gap. In general, however, a narrower temperature gap will require more stringent annealing conditions in the assembly step to provide the requisite level of fidelity. Practically, the difference between the lowest-melting correct match and the highest melting incorrect match is at least about 1° C., more preferably, at least about 4° C., still more preferably, at least about 8° C., still more preferably, at least about 16° C. In general, the wider the temperature gap, the more robust the self-assembly, thereby permitting the use of less stringent annealing conditions.

FIG. 2A schematically illustrates the first hierarchical division step. A desired gene 2000 is divided into a plurality of overlapping intermediate fragments 2100, 2200, 2300, 2400, 2500, and 2600. FIGS. 2A-2F illustrate exemplary division and reassembly steps using six fragments in each step. Those skilled in the art will understand that some or all steps in other embodiments use greater or fewer than six fragments, and in some preferred embodiments, many more than six fragments. Those skilled in the art will also understand that the number of fragments is odd or even. As discussed in detail in U.S. Patent Publication Nos. 2004/0235035 A1 and 2005/0106590 A1, some preferred embodiments using an even number of fragments and PCR-type assembly do not use PCR primers. As discussed above, in some preferred embodiments, the gene 2000 is one of a plurality of genes in a pool, all of which undergo simultaneous division and design, and assembly, as described herein.

In step 104, the oligonucleotides or intermediate DNA fragments at each hierarchical level are merged into a plurality of pools. In some preferred embodiments, the pools are maximal pools. As used herein, the term “maximal pool” is a broad term that refers to a pool of DNA fragments comprising only non-overlapping pieces of DNA resulting from the division in step 102, and results from a division in step 102 into a number of pools that is less than or equal to the number of pools resulting from any other possible division in step 102. For biologically reasonable DNA sequences, those skilled in the art will understand that the fragments resulting from the division of a linear piece of DNA as described in step 102 are assignable into two maximal pools. Fragments resulting from the division of a circular piece of DNA are assignable into at most three maximal pools; however, those skilled in the art will understand that a division according to step 102 exists in which the fragments are assignable into two maximal pools.

In each maximal pool created in step 104, the next-larger pieces of DNA produced by the oligonucleotides or intermediate fragments in that pool do not overlap. For example, in a two-level synthesis of a synthetic gene, the gene is divided into intermediate fragments, which are in turn divided into oligonucleotides. In some embodiments, the intermediate fragments are divided into two pools of non-overlapping intermediate fragments. For convenience, the fragments are referred to herein as “odd numbered” or “odd” fragments, and “even numbered” or “even” fragments, referring to their order in the assembled synthetic gene. Those skilled in the art will understand that in some embodiments, one or more of the pools comprises both odd and even fragments derived from non-adjacent pieces of DNA, for example, the odd fragments derived from one larger piece of DNA and the even fragments derived from another non-overlapping piece of DNA. In other embodiments, none of the pools comprises both odd and even fragments derived from non-adjacent pieces of DNA. Because only adjacent intermediate fragments designed in step 102 overlap each other, the even and odd numbered fragment pools are each composed internally of non-overlapping intermediate fragments. In step 104, after the final hierarchical division step, a first pool is created from the oligonucleotides that produce the odd intermediate fragments, and a second pool is created from the oligonucleotides that produce the even intermediate fragments. After the intermediate fragments are produced from the oligonucleotides, the resulting pooled even intermediate fragments are combined with the resulting pooled odd intermediate fragments to form a pool of DNA pieces that produces the next larger intermediate fragments at the next higher hierarchical level, or the desired synthetic gene(s) in the final step.

Using oligonucleotides as an example, because the sequences of the oligonucleotides are optimized to produce the intermediate fragments with a high thermodynamic probability, separating the oligonucleotides that form adjacent, overlapping intermediate fragments eliminates undesired interactions between the oligonucleotides that comprise the overlapping fragments, and which may lead to undesired products. Those skilled in the art will understand that step 104 also encompasses embodiments in which the pools are not maximal, that is, in which more than two pools of DNA pieces are used at a hierarchical level.

Returning to FIG. 2A, the fragments 2100, 2200, 2300, 2400, 2500, and 2600 are divided into two maximal pools: an odd fragment pool 2000a comprising the odd numbered fragments 2100, 2300, and 2500; and an even fragment pool 2000b comprising the even numbered fragments 2200, 2400, and 2600.

In step 106, steps 102 and 104 are repeated for each pool until the resulting pieces of DNA, i.e., oligonucleotides, in each pool is easily obtainable, for example, by chemical synthesis.

FIG. 2B illustrates an intermediate hierarchical division step 102 in which each of the intermediate fragments 2100, 2200, 2300, 2400, 2500, and 2600 is divided into a plurality of overlapping fragments. Only the division of fragment 2100 into fragments 2110, 2120, 2130, 2140, 2150, and 2160 is illustrated.

In repeated step 104 the overlapping fragments created in repeated step 102 are merged into maximal pools. As illustrated in FIG. 2B, the fragments 2110, 2120, 2130, 2140, 2150, and 2160 are assigned to an odd fragment pool 2000aa comprising the odd numbered fragments 2110, 2130, and 2150; and an even fragment pool comprising the even numbered fragments 2120, 2140, and 2160. Those skilled in the art will appreciate that the odd fragment pool 2000aa comprises all of the odd fragments created in the division of all of the fragments of the odd fragment pool 2000a, that is, of fragments 2100, 2300, and 2500. Similarly, the even fragment pool 2000ab comprises all of the even fragments created in the division of the fragments of all of the odd fragment pool 2000a. Although not illustrated in detail in FIG. 2B, steps 102 and 104 are also repeated on even fragment pool 2000b from FIG. 2A, resulting in the odd fragment pool 2000ba and the even fragment pool 2000bb, for a total of four pools. Those skilled in the art will understand that in some embodiments, each hierarchical level n comprising a division step 102 and a merging step 104 results in 2″ maximal pools. In other embodiments, divided fragments from one or more of the pools are not further merged into new pools, resulting in more or fewer than 2″ pools.

As discussed above, the terms “odd” and “even” are used for convenience only to indicate non-overlapping intermediate fragments. Accordingly, in some embodiments, the odd fragment pool comprises the odd fragments 2110, 2130, and 2150 created in the division of intermediate fragment 2100 as well as the even fragments created in the division of another non-overlapping intermediate fragment in the same pool as fragment 2100, for example, fragment 2500.

FIG. 2C illustrates a final hierarchical division step 102 for fragment 2110 from odd pool 2000aa into the oligonucleotides 2111, 2112, 2113, 2114, 2115, and 2116. As discussed above, in a final division, step 104 in not repeated, and consequently, the resulting oligonucleotide pool 2000aa′ comprises all of the oligonucleotides created in the division of the fragments 2110, 2130, and 2150 from pool 2000aa, together with the oligonucleotides created in the division of all other fragments from pool 2000aa. Similarly, pools 2000ab′, 2000ba′, and 2000bb′ are created through application of the division step 102 to the pools 2000ab, 2000ba, and 2000bb, respectively.

Those skilled in the art will understand that in some preferred embodiments, one or more of the steps in the division phase, steps 102, 104, and/or 106, are automated, for example, using a data processing unit, computer, microprocessor, purpose-built device, and/or other suitable machine known in the art. In some preferred embodiments, all of these steps are automated. In some preferred embodiments, machine readable instructions that, when executed, perform the automated steps are stored on any machine readable media known in the art, for example, magnetic media, optical media, magneto-optical media, phase-change media, solid state media, combinations thereof, and the like. Particular examples of suitable media include magnetic disks, magnetic tapes, optical disks, flash memory, and the like. In some embodiments, the machine readable media is remote from the user, for example, on one or more servers that the user accesses using one or more networks.

In step 110, pools of synthetic oligonucleotides are obtained. In some preferred embodiments, all of the oligonucleotides are synthetic. In other embodiments, at least one oligonucleotide is not synthetic, for example, derived from a natural source, for example, using one or more restriction enzymes. The synthetic oligonucleotides are from any suitable source, including, for example, oligonucleotides synthesized individually on a solid-phase support(s), oligonucleotides cleaved from DNA chips, and the like. In some embodiments, the pooled oligonucleotides are optionally purified as discussed below in step 116, for example, by filtration and/or by electrophoresis.

The pooled oligonucleotides are from any source known in the art. In some embodiments, the pooled oligonucleotides are synthesized combinatorially. In some embodiments, the pooled oligonucleotides are synthesized on beads, for example, as reported in U.S. Pat. No. 5,808,022. In other embodiments, the pooled oligonucleotides are synthesized on an array or microarray, for example, as disclosed in U.S. Patent Publication No. 2004/0068633 A1; in Tian et al., Nature, 2004, 432, 1050-1054; and in Richmond et al., Nucleic Acids Res., 2004, 32(17), 5011-5018. The syntheses of some embodiments of pooled synthetic oligonucleotides are highly efficient, and consequently, these oligonucleotide pools are relatively inexpensive.

Referring to FIG. 2C, pools of DNA oligonucleotides corresponding to pools 2000aa′, 2000ab′, 2000ba′, and 2000bb′ created in step 106 are obtained and optionally filtered.

In step 112, the oligonucleotides in each pool are allowed to self-assemble into DNA constructs. As discussed above, in step 104, the pieces of DNA from each parent pool at the next-higher hierarchical level are assigned into two new pools at the next-lower level: one pool containing the even numbered intermediate fragments, and the other pool containing the odd numbered intermediate fragments, of that parent pool. As discussed above, the even and odd numbered fragment pools designed in step 104 are each composed internally of non-overlapping intermediate fragments. Thus, each intermediate round of amplification will not extend beyond intermediate fragment boundaries until the final assembly step, which produces one or more full-length genes. This property reduces the assembly complexity at each hierarchical step, thereby reducing the likelihood of incorrect assembly (mis-priming or cross-hybridization). In some embodiments, all intermediate fragments in a pool have the same or about the same length, which permits more efficient purification, as discussed below.

FIG. 21D illustrates the self-assembly of the oligonucleotides 2111, 2112, 2113, 2114, 2115 into a DNA construct 2110′. As discussed above, the pool 2000aa′ of oligonucleotides, comprises all of the oligonucleotides created in step 102 and illustrated in FIG. 2C for the fragments in pool 2000aa. Accordingly, DNA constructs corresponding to the other DNA fragments in pool 2000aa, that is, fragments 2130 and 2150 and the odd fragments of fragments 2300 and 2500, are also formed in this step. Corresponding DNA constructs are also formed in pools 2000ab′, 2000ba′, and 2000bb′.

In step 114, the next-larger pieces of DNA are produced from the DNA constructs formed in step 112 using any method known in the art, for example, as disclosed in U.S. Patent Publication Nos. 2004/0235035 A1 and 2005/0106590 A1. Briefly, in some embodiments, the next-larger pieces of DNA are produced using overlap extension using appropriate primers. In some preferred embodiments, step 114 comprises a high-fidelity DNA polymerase reaction. In some embodiments with no gaps between the double-stranded overlaps, the next-larger pieces of DNA are produced by ligation. In some embodiments, the self-assembled construct(s) are cloned into an expression vector, and the next-larger pieces of DNA are produced by the cellular machinery. Some preferred embodiments use overlap extension, which is also referred to herein as PCR-type assembly, or simply, PCR.

In some embodiments, one or more of the product next-larger pieces of DNA are also amplified in this step. In embodiments in which a single synthetic gene is selectively synthesized from pools comprising oligonucleotides designed for the synthesis of a plurality of synthetic genes, step 114 uses primers specific for the synthesis of the desired synthetic gene.

FIG. 2D) illustrates the assembly of the DNA constructs formed in step 112 in pool 2000aa′ to provide pool 2000aa, which comprises fragments 2110, 2130, and 2150. As illustrated, overlap extension of the construct 2110′, which comprises the oligonucleotides 2111, 2112, 2113, 2114, 2115, and 2116, forms fragment 2110. Accordingly, the resulting pool 2000aa comprises the fragments 2110, 2130, and 2150, and the odd fragments of fragments 2300 and 2500. Overlap assembly of the constructs in pools 2000ab′, 2000ba′, and 2000bb′ provide pools 2000ab, 2000ba, and 2000bb, respectively.

In optional step 116, errors one or more of the next-larger pieces of DNA are reduced, and/or quantities of pieces of DNA are increased, using any suitable method known in the art, for example, by purification and/or amplification. Purification and/or amplification is performed by any suitable means known in the art. Examples of suitable purification methods include enzymatic, electrophoretic, and/or chromatographic methods. Chromatographic purification is also referred to herein as “filtering.” Examples of suitable amplification methods include PCR. In some preferred embodiments, all of the pieces of DNA formed from a pool in step 114, for example, the resulting reaction mixture, are subjected to the purification and/or amplification conditions.

Some embodiments of enzymatic purification use T7 endonuclease, which cleaves mismatched DNA. Some embodiments of enzymatic purification use MutS, for example, immobilized on magnetic beads, to repair mismatches and other errors. Some embodiments of enzymatic purification use immobilized DNA glycosylases, for example, as disclosed in U.S. Patent Publication No. 2003/0134289 A1. In some embodiments, the next-larger pieces of DNA are purified chromatographically and/or electrophoretically, for example, using size exclusion chromatography, gel permeation chromatography (GPC), molecular-sieve chromatography, high-performance liquid chromatography (HPLC), fast protein liquid chromatography (FPLC), polyacrylamide gel electrophoresis (PAGE), capillary electrophoresis, agarose electrophoresis, combinations thereof, and the like. In some preferred embodiments, the purification comprises PAGE.

In some preferred embodiments, the next-larger pieces of DNA in each pool are designed to have the same size or nearly the same size. Accordingly, all of the correct and nearly-correct next-larger pieces of DNA run as a single band on a gel or column. In some embodiments, the DNA from this band is extracted and forms the pool used in step 118. Optionally, DNA produced in step 114 is amplified, for example, using PCR. Amplification is performed at any time in any stage of step 116, for example, before and/or after enzymatic purification, for example, treating with T7 and/or MutS. In some embodiments, amplification is performed before and/or after chromatographic and/or electrophoretic purification.

In some embodiments, the pieces of DNA produced in a pool are purified using a method that comprises a plurality of purification methods, for example, an enzymatic method followed by an electrophoretic method. Those skilled in the art will recognize that other filtering and/or purification methods are used in other embodiments.

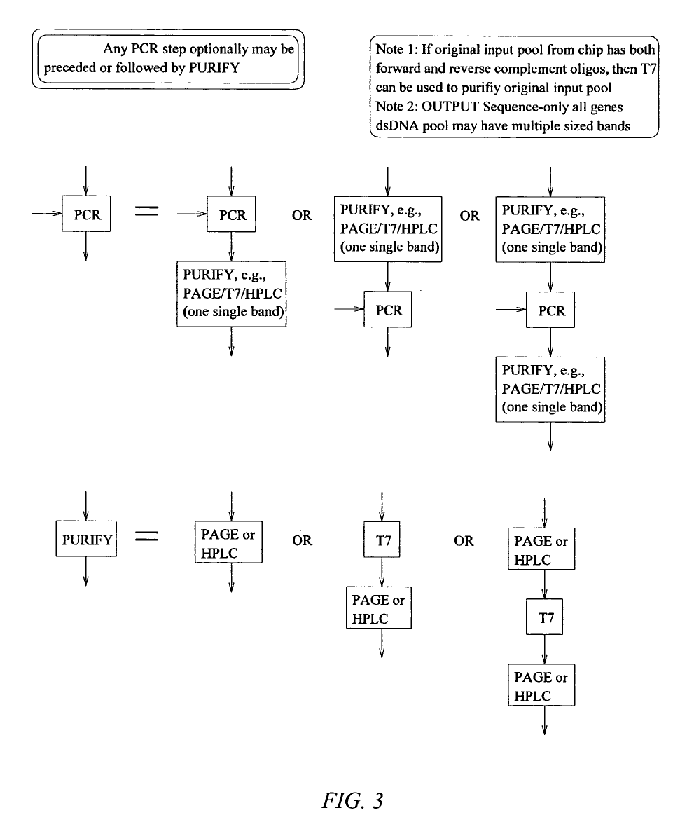

FIG. 3 illustrates embodiments of step 116 comprising different combinations of producing the next-larger piece of DNA in step 114 by polymerase overlap extension (PCR), and purification in step 116 by enzymatic, chromatographic, and/or electrophoretic techniques. In the illustrated embodiments, overlap extension 114 is performed before, after, and/or between purification steps 116. Embodiments of the purification include combinations of enzymatic and chromatographic/electrophoretic methods. Those skilled in the art will understand that other combinations are possible. Those skilled in the art will also understand that, in some embodiments, other methods for carrying out step 114, for example, ligation, are used in place of the overlap extension illustrated in FIG. 3.

Purification and/or amplification is not illustrated in FIGS. 2D-2F, but is used in at least once in some embodiments, for example, after one or more of the hierarchical assembly steps.

In step 118, the DNA pools for the following hierarchical synthesis level are created by combining the pools of DNA produced in step 114 and/or 116. In some embodiments, purification and/or amplification step 116 is performed after the DNA pools are created in step 118.

FIG. 2E illustrates the creation of pool 2000a′ from pools 2000aa and 2000ab. Similarly, pool 2000b′ is created from pools 2000ba and 2000bb. Pool 2000a′ comprises fragments 2110, 2120, 2130, 2140, 2150, and 2160, as well as DNA fragments corresponding to the division of intermediate fragments 2300 and 2500 in step 106. Pool 2000b′ comprises DNA fragments corresponding to the division of intermediate fragments 2200, 2400, and 2600 in step 106.

In step 120, steps 114, 116, and 118 are repeated for each hierarchical level to assemble of the synthetic gene or genes.

FIG. 2E illustrates the self-assembly and overlap extension of pool 2000a′ in repeated steps 112 and 114 to provide pool 2000a, which comprises the intermediate fragments 2100, 2300, and 2500. Intermediate fragment 2100 exemplifies the assembly of all of the intermediate fragments in pool 2000a. Pool 2000b is assembled from pool 2000b′ similarly.

FIG. 2F illustrates the creation of pool 2000′ from pools 2000a and 2000b in repeated step 118, and self-assembly and overlap extension in repeated steps 112 and 114 to provide synthetic gene 2000. In the illustrated embodiment, the final overlap extension step 114 is gene specific, which permits the synthesis of a single gene from pools designed to produce a plurality of genes. In some embodiments, gene specific overlap extension comprises using one or more PCR primers that are designed to anneal to only the desired gene, thereby selectively amplifying that gene.

In step 130, one or more synthetic genes are isolated and/or purified from the products of the final iteration of step 120, by any means known in the art, for example, by electrophoresis (e.g., PAGE) and/or by PCR using appropriate primers. In some embodiments, step 130 is optional. Some preferred embodiments of step 130 are disclosed in U.S. Patent Publication No. 2005/0106590 A1.

In step 132, one or more synthetic genes with the correct sequences is selected by any suitable method known in the art. In some preferred embodiments, selection uses a frameshift vector and sequencing, for example as disclosed in U.S. Patent Publication No. 2005/0106590 A1.

Briefly, the full-length gene is selected by a method comprising at least the steps of: (i) inserting the full-length gene into a DNA insertion site in a frameshifted vector; (ii) transforming a preselected organism with the resulting vector; (iii) selecting an organism exhibiting a predetermined phenotype; and (iv) isolating the full-length gene from the selected organism. The frameshifted vector comprises an open reading frame comprising an indicator gene and the DNA insertion site. The indicator gene comprises a functional portion that encodes a functional polypeptide, the expression of which changes the phenotype of the organism. The functional portion of the indicator gene is frameshifted relative to an upstream start codon such that no functional polypeptide is expressed. In some embodiments, the frameshifted vector comprises the start codon. In some embodiments, the start codon is designed into the synthetic gene. The DNA insertion site is upstream of the functional portion of the indicator gene.

A correct full-length gene, when inserted at the DNA insertion site with additional bases that correct the frameshift causes the functional portion of the indicator gene to express a functional polypeptide. An incorrect full-length gene containing one or two base insertions or deletions, or any number of base insertions and deletions whose sum is not an even multiple of three (i.e., not congruent to 0 (mod 3)), will fail to cause the indicator gene to express a functional polypeptide. Those skilled in the art will understand that a +2 frame shift is equivalent to a −1 frameshift, and a −2 frameshift is equivalent to a +1 frameshift.

In some embodiments, the frameshifted vector is a plasmid, and the preselected organism is E. coli. In some preferred embodiments, the plasmid comprises a gene for the alpha-complementing fragment of beta-galactosidase, and the preselected organism is an E. coli strain with the lacZ-delta-M15 genotype. In some embodiments, the transformed E. coli is grown on indicator agar comprising isopropylthio-beta-D-galactoside (IPTG) and 5-bromo-4-chloro-3-indolyl-beta-D-galactoside (X-Gal), and wherein the predetermined phenotype is a blue colored colony. In some preferred embodiments, the plasmid has SEQ. ID. NO.: 1, which is a frameshifted vector useful in the lacZ-delta-M 15 system, and the preselected organism is E. coli JM109. In other embodiments, change in phenotype is growth at a restrictive temperature. In some embodiments, the plasmid comprises a gene for valyl-tRNA synthesase^{ts} and the preselected organism is E. coli AB4141. In some preferred embodiments, the plasmid has SEQ. ID. NO.: 2, which is a frameshifted vector useful in the valyl-tRNA synthesase^{ts} system. In some embodiments, the frameshift is a −1 frameshift. In other embodiments, the frameshift is a +1 frameshift. In some embodiments, the DNA insertion site comprises a restriction site.

Those skilled in the art will understand that other suitable methods for selecting a synthetic gene with the correct sequence are also useful. In some embodiments, step 132 is optional.

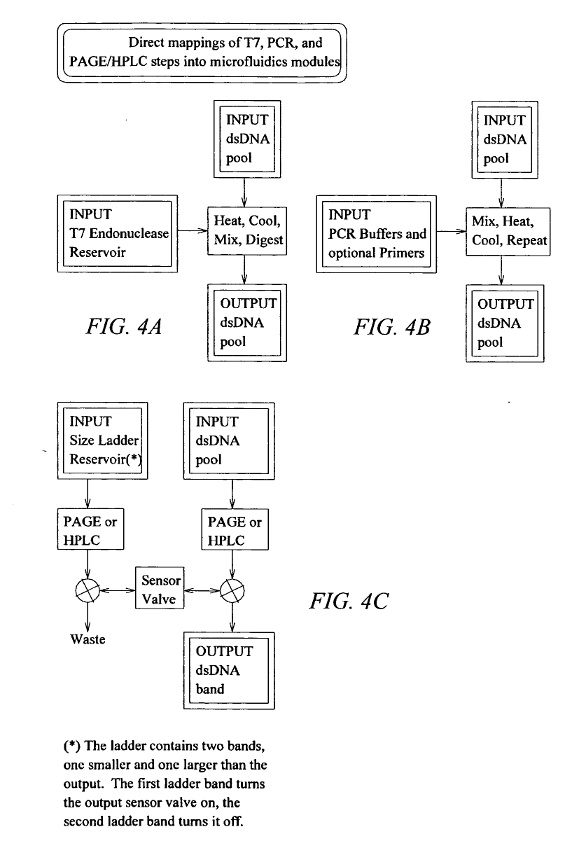

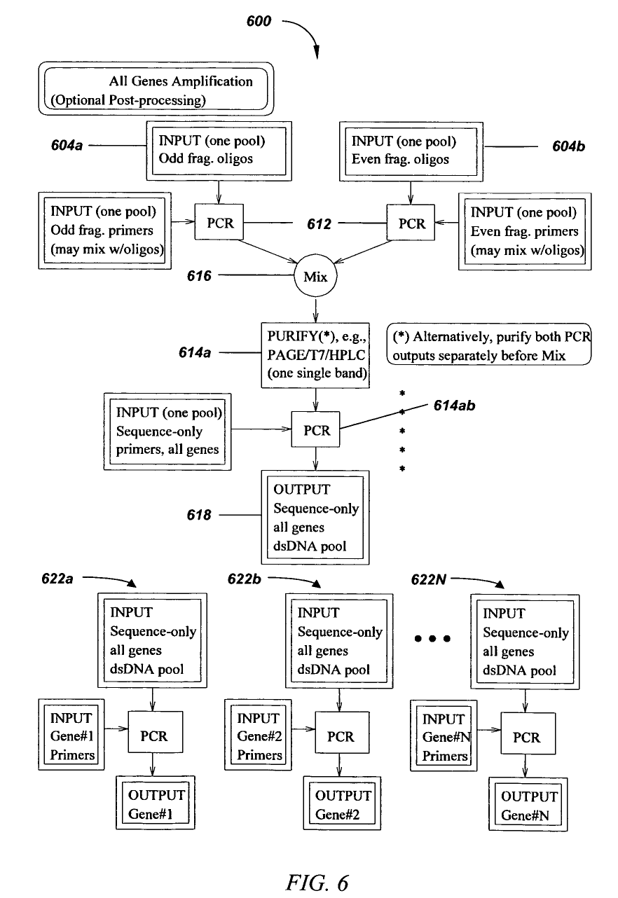

In some embodiments, any or all of the steps in the method 100 are implemented using one or more automated systems. Some embodiments use automated systems, for example, robots, automated fluid handling systems, combinations thereof, and the like, for performing any or all of steps 112, 114, 116, and/or 118. Typically, such systems are under computer and/or microprocessor control. FIGS. 4A-C illustrate schematically the purification and amplification of the next-larger pieces of DNA, for example, step 116 of method 100, using microfluidics modules of any suitable type known in the art. FIG. 4A schematically illustrates a purification of a DNA fragment pool synthesized in step 114 using T7 endonuclease. FIG. 4B schematically illustrates amplification of a DNA fragment pool by PCR. FIG. 6C illustrates chromatographic or electrophoretic purification of a DNA fragment pool. Those skilled in the art will understand that other steps in the disclosed method are also amenable to automation, for example, the synthesis of pooled oligonucleotides, extension of DNA constructs to produce the next-larger pieces of DNA, creating pools of DNA for the next hierarchical synthesis level, PCR isolation of a desired synthetic gene, cloning into a frame shifted vector, and the like.

Also provided is a synthetic gene synthesis kit. The kit comprises a plurality of pools of oligonucleotides, wherein the composition of each oligonucleotide pool is determined by division, optimization, and merging as described above in steps 102, 104, and 106 of method 100. In some preferred embodiments, the hierarchical division is a two-level division, and the gene synthesis kit comprises three components: an odd pool of oligonucleotides, an even pool of oligonucleotides, and PCR primers for amplifying a first full length gene. Some embodiments of the oligonucleotide pools further comprise suitable primers known in the art for the overlap extension reactions of the oligonucleotides and/or intermediate fragments. Those skilled in the art will understand that other embodiments of synthetic gene synthesis kit use higher levels of hierarchical division, for example, from 3 to about 5, and consequently, comprise additional oligonucleotide pools, as discussed above.

As discussed above, in some embodiments, steps 102, 104, and 106 of method 100 are simultaneously applied on a mixture of a plurality of genes, thereby resulting in oligonucleotide pools that encode their own correct self-assemblies into the mixture of the plurality of genes through the application of steps 112, 114, 116, 118, and 120. Accordingly, some embodiments of the gene synthesis kit comprise one or more additional sets of primers useful for amplifying a second gene and/or additional genes from the final overlap extension reaction product mixture. In some preferred embodiments, each oligonucleotide pool and/or set of primers is conveniently supplied in a suitable tube or container of any type known in the art, for example, a microcentrifuge tube or wells in a microtiter plate, for example, commercially available from Eppendorf (Hamburg, Germany). In some preferred embodiments, one or more of the oligonucleotide pools and/or set of primers is supplied in the solid state.

Gene synthesis kits not using the disclosed method typically use one tube comprising the oligonucleotides needed to assemble each intermediate DNA fragment, plus a tube of PCR primers for each intermediate DNA fragment. Embodiments of three-tube synthetic gene synthesis kit exhibit one or more advantages, including, cost savings and/or labor efficiencies.

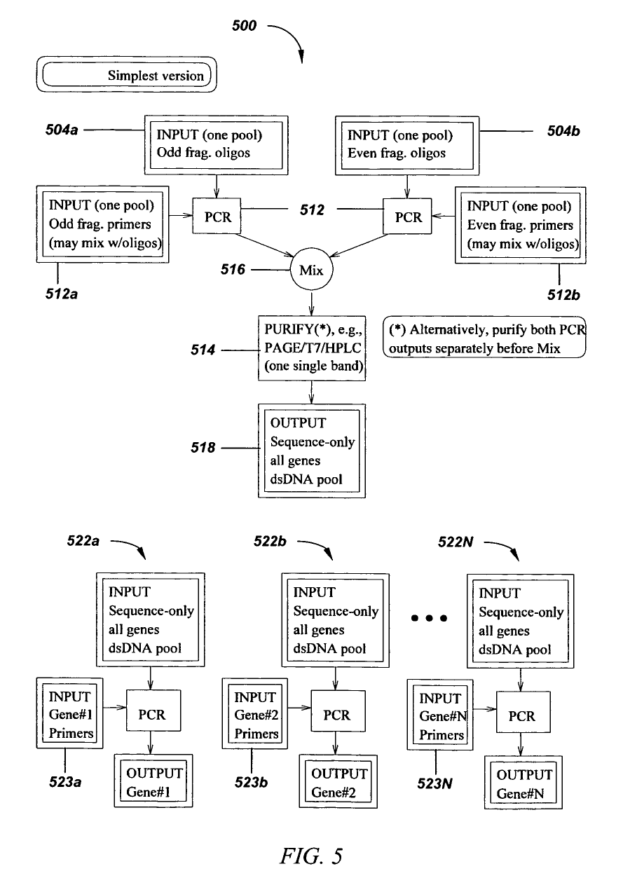

An embodiment of a method 500 for synthesizing a synthetic gene from the disclosed two-level gene synthesis kit is illustrated schematically in FIG. 5. In the illustrated embodiment, sequences of N synthetic genes are divided, optimized, and merged as described above in steps 102, 104, and 106 to provide an oligonucleotide pool 504a designed for the synthesis of the odd intermediate fragments and a pool 504b designed for the synthesis of the even intermediate fragments. The corresponding pools are obtained and mixed with the appropriate PCR primers 512a and 512b. The odd and even intermediate fragments are synthesized by PCR 512. The resulting intermediate fragments are mixed to form the next higher pool of DNA 516. The resulting pool is purified in step 514. In the illustrated embodiment, all of the intermediate fragments have about the same length. Consequently, electrophoresis (e.g., PAGE) of the mixture provides a single band for the pooled intermediate fragments. Completing the synthesis provides a mixture of N synthetic genes 518. Each of the N genes is isolatable from the mixture 518 using PCR in processes 522a-522N using the appropriate primers 523a-523N.

Another embodiment of the method 600 is illustrated in FIG. 6. Odd and even intermediate fragments are synthesized by PCR 612 from their respective oligonucleotide pools 604a and 604b. Mixing the odd and even intermediate fragments provides a pool of intermediate fragments 616, which is purified in two steps in the illustrated embodiment. In step 614a, the intermediate fragments are purified using a combination of T7, PAGE, and/or HPLC. In step 614b, all of the intermediate fragments are amplified by PCR. A mixture 618 of N synthetic genes is produced from resulting amplified pool. Synthetic genes 1 through N are isolated from the mixture 618 by PCR in steps 622a-622N using the appropriate primers.

### EXAMPLE

### Yeast Ty3 1N Gene for Expression in E. coil Recursive Decomposition and Overlap Extension Assembly

The yeast Ty3 integrase (Ty3 1N) gene is a 1640 bp gene from Saccharomyces cerevisiae. The gene was divided, optimized and merged to provide 15 199 bp intermediate fragments in a first hierarchical division, which were in turn divided into 90 oligonucleotides in a second hierarchical division. As discussed above, each of the 90 oligonucleotides and 15 intermediate DNA fragments were designed to hybridize only to adjacent overlapping pieces of DNA, and to avoid globally all incorrect hybridizations to all other oligonucleotides and/or intermediate DNA fragments. The overlaps between adjacent intermediate fragments was from 77 bp. he overlaps between adjacent oligonucleotides was from 25 bp to 26 bp. The melting point gap between the lowest melting correct hybridization and the highest melting incorrect hybridization was 19.8° C.

The optimized sequence of the Ty3 1N gene has SEQ. ID. NO.: 3. The gene leader and gene trailer (reverse complement strand) for the intermediate fragments have SEQ. ID. NO.: 4 and SEQ. ID. NO.: 5, respectively. Each of the 15 intermediate fragments is identified as “Fragment x,” where x is 0-14, with SEQ. ID. NO.: 6-SEQ. ID. NO.: 20, respectively. Each of the 90 oligonucleotides is identified as “Oligo-x-y,” where x is as defined above, and y is 0-5, with SEQ. ID. NO.: 21-SEQ. ID. NO.: 110, respectively. Each of the sequences where y is odd is the reverse complement strand. The odd pool of oligonucleotides comprised the 42 oligonucleotides where x is odd. The even pool of oligonucleotides comprised the 48 oligonucleotides where x is even.

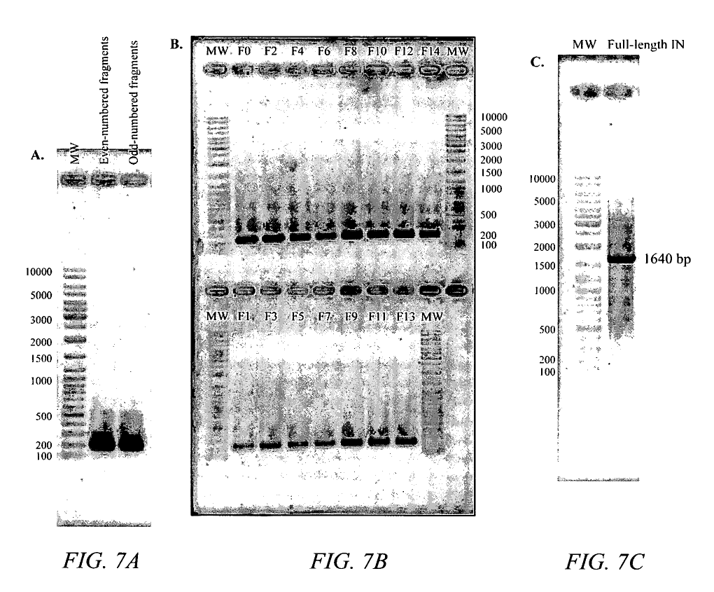

The odd and even pools were prepared using oligonucleotides purchased from a commercial source (Integrated DNA Technologies (IDT), Coralville, Iowa). The final concentrations of each of the 42 oligonucleotides in the odd pool (x odd) was 2 μM, except the first and the last oligos for each fragment (Oligo-x-0 and Oligo-x-5), which were at 10 μM. The final concentration of each of the 48 oligonucleotides in the even pool (x even) was 2 μM, except the first and the last oligos of each fragment (Oligo-x-0 and Oligo-x-5), which were at 10 μM. Two parallel PCR reaction mixtures were prepared using 2.8 μL or 3.2 μL of the odd or even oligonucleotides pool, respectively; 1 μL of 10 mM dNTPs; 5 μL of 10× PfuUltra™ II polymerase buffer; and 1 μL (2.5 Units/μL) of PfuUltra™ II polymerase diluted to a final volume to 50 μL with distilled H₂O. PfuUltra™ II is a high-fidelity fusion DNA polymerase commercially available from Stratagene (La Jolla, Calif.). The PCR reactions were performed in a MJ Research PTC225 Thermocycler using the following calculated-control protocol: 10 min denaturation step at 95° C.; followed by 25 cycles of 20 sec at 95° C., 30 sec at 60° C., and I min at 72° C.; and a final step of 5 min at 72° C. Because each of the correctly assembled fragments is 199 bp in length, electrophoresis on a 1.2% agarose gel provided 1 major band as illustrated in FIG. 7A.

In order to confirm that the assembly of each intermediate fragment proceeded as designed, 15 parallel PCR reactions were performed on the product reaction mixtures, each using the appropriate first and last oligonucleotides (oligo-x-0 and oligo-x-5) for each fragment as primers. Each reaction mixture used 1 μL of the PCR products of the odd pool or even pool, as appropriate; 1 μL of the PCR primers from a solution of 10 μM of each; 1 μL of 10 mM dNTPs, 5 μL of 10× PfuUltra™ II polymerase buffer; and 1 μL (2.5 Units/μL) of PfuUltra™ II polymerase diluted to a final volume to 50 μL with distilled H₂O. The PCR reactions were performed in a MJ Research PTC225 Thermocycler using the following calculated-control protocol: 10 min denaturation step at 95° C.; followed by 25 cycles of 20 sec at 95° C., 30 sec at 60° C., and 30 sec at 72° C.; and a final step of 5 min at 72° C. Agarose gel electrophoresis analysis confirmed the correct assembly of the 15 199-bp intermediate fragments, as illustrated in FIG. 7B.

Next, 0.7 pmole of the combined odd number fragments and 0.8 pmole of the combined even number fragments were mixed together with 1 μL of a gene primers leader and trailer mix (mixture of the gene leader and gene trailer at 10 μM each final), 1 μL of 10 mM dNTPs, 5 μL of 10× PfuUltra™ II polymerase buffer; and 1 μL (2.5 Units/μL) of PfuUltra™ II polymerase diluted to a final volume to 50 μL with distilled H₂O. The PCR reaction was performed in a MJ Research PTC225 Thermocycler using the following calculated-control protocol: 10 min denaturation step at 95° C.; followed by 25 cycles of 20 sec at 95° C., 30 sec at 68° C., and 2 min at 72° C.; and a final step of 5 min at 72° C. The resulting product was analyzed by electrophoresis on a 1.2% agarose gel, which showed a single band of the correct size (1640 bp), as illustrated in FIG. 7C.

The embodiments illustrated and described above are provided as examples of certain preferred embodiments. Various changes, modifications, substitutions can be made to the embodiments presented herein by those skilled in the art without departure from the spirit and scope of this disclosure, the scope of which is limited only by the claims appended hereto.

Those skilled in the art will understand that changes in the method and/or system described above are possible, for example, adding and/or removing components and/or steps, and/or changing their orders. While the above detailed description has shown, described, and pointed out novel features as applied to various embodiments, it will be understood that various omissions, substitutions, and changes in the form and details of the method and/or system illustrated may be made by those skilled in the art without departing from the spirit of this disclosure. As will be recognized, some embodiments do not provide all of the features and benefits set forth herein, and some features may be used or practiced separately from others.

What is claimed is:

1. A method for hierarchically synthesizing a piece of DNA with a desired nucleic acid sequence, the method comprising a hierarchical division of a nucleic acid sequence of a piece of DNA with a desired nucleic acid sequence by a method comprising:
(i) hierarchically dividing the nucleic acid sequence into a plurality of DNA sequences, wherein adjacent DNA sequences comprise overlapping portions;
(ii) optionally, optimizing at least some of the DNA sequences to strengthen correct hybridizations between the overlapping portions of adjacent DNA sequences and to weaken incorrect hybridizations;
(iii) assigning, at each hierarchical level of division except a final hierarchical level of division, the DNA sequences into a plurality of pools of DNA sequences, wherein adjacent DNA sequences with overlapping portions are assigned to different pools; and
(iv) recursively repeating steps (i), (ii), and (iii) for each DNA sequence in each pool.

2. The method of claim 1, wherein
the piece of DNA with a desired nucleic acid sequence is a member of a pool comprising a plurality of pieces of DNA with desired nucleic acid sequences, and
the hierarchical division is simultaneously performed on the nucleic acid sequences of the plurality of pieces of DNA with desired nucleic acid sequences.

3. The method of claim 1, wherein in at least one hierarchical level of division, all of the DNA sequences are about the same size.

4. The method of claim 1, wherein the method comprise optimizing within at least one hierarchical level of division, and the optimizing comprises globally optimizing all possible correct and incorrect hybridizations between every DNA sequence in at least one pool.

5. The method of claim 1, wherein the method comprise optimizing within at least one hierarchical level of division, and the optimizing comprises calculating a temperature gap between a melting temperature of a lowest correct hybridization and a melting temperature of a highest incorrect hybridization.

6. The method of claim 5, wherein the temperature gap is at least about 1° C.

7. The method of claim 1, wherein the method comprises optimizing within at least one hierarchical level of division, and optimizing comprises permuting a silent codon substitution.

8. The method of claim 7, wherein the silent codon substitution is a substitution according to a codon usage preference for an organism.

9. The method of claim 8, wherein the codon usage preference is a codon pair preference.

10. The method of claim 8, wherein the organism is E. coli.

11. The method of claim 1, wherein the method comprises optimizing within at least one hierarchical level of division, and optimizing comprises taking advantage of a degeneracy in a regulatory region consensus sequence.

12. The method of claim 1, wherein the method comprises optimizing within at least one hierarchical level of division, and optimizing comprises adjusting boundary points between adjacent resulting pieces of DNA.

13. The method of claim 1, wherein the optimizing in at least one hierarchical level of division comprises direct base assignment.

14. The method of claim 1, wherein at least one of the pools comprises at least some of the DNA sequences resulting from a division of a plurality of next-larger DNA sequence from a next-higher hierarchical level of division.

15. The method of claim 1, wherein the pools are maximal pools.

16. The method of claim 1, wherein the method is automated.

17. A method for hierarchically synthesizing a piece of DNA with a desired nucleic acid sequence, the method comprising a hierarchical assembly of a piece of DNA with a desired nucleic acid sequence by a method comprising:
(v) obtaining pools of pieces of DNA corresponding to pools of DNA sequences of a final hierarchical division produced according to the method of claim 1 performed on a nucleic acid sequence of the piece of DNA with a desired nucleic acid sequence;
(vi) allowing the pieces of DNA in each pool to self-assemble into DNA constructs corresponding to next-larger pieces of DNA in a next-higher hierarchical level of division;
(vii) producing the next-larger pieces of DNA from the DNA constructs;
(viii) creating pools of the next-larger pieces of DNA corresponding to the next-higher hierarchical level of the division; and
(ix) recursively repeating steps (vi), (vii), and (viii) in the reverse order of the hierarchical division in steps (i), (ii), (iii), and (iv) to synthesize the piece of DNA with a desired nucleic acid sequence.

18. The method of claim 17, wherein the pieces of DNA in step (v) are synthetic oligonucleotides.

19. The method of claim 17, wherein in at least one hierarchical level of assembly, the next-larger pieces of DNA are about the same size.

20. The method of claim 17, wherein in at least one hierarchical level of assembly, producing the next-larger pieces of DNA comprises polymerase overlap extension or ligation.

21. The method of claim 20, wherein in at least one hierarchical level of assembly,
producing the next-larger pieces of DNA comprises polymerase overlap extension; and
the polymerase overlap extension comprises a high-fidelity DNA polymerase reaction.

22. The method of claim 17, further comprising in at least one hierarchical level of assembly, at least one of purifying or amplifying the next-larger pieces of DNA after at least one of steps (vii) or (viii).

23. The method of claim 22, wherein the purifying comprises at least one of electrophoresis or chromatography.

24. The method of claim 22, wherein the purifying comprises treatment with an enzyme.

25. The method of claim 24, wherein the enzyme is MutS, T7 endonuclease, or a combination thereof.

26. The method of claim 22, wherein the amplifying comprises a polymerase chain reaction.

27. The method of claim 17, wherein at least one of steps (vi), (vii), or (viii) is automated.

28. The method of claim 27, wherein at least one of the automated steps is performed microfluidically.

29. The method of claim 17, wherein
the piece of DNA with a desired nucleic acid sequence is a member of a pool comprising a plurality of pieces of DNA with desired nucleic acid sequences, and
the pools of pieces of DNA in step (v) correspond to pools of DNA sequences of a final hierarchical division produced according to the method of claim 1 performed on the nucleic acid sequences of the plurality of pieces of DNA with desired nucleic acid sequences.

30. The method of claim 29, wherein the product of the final hierarchical assembly comprises a pool of pieces of DNA with desired sequences.

31. The method of claim 17, further comprising isolating at least one piece of DNA with a desired sequence after the last hierarchical assembly step.

32. The method of claim 30, wherein the isolating comprises a polymerase chain reaction.

33. The method of claim 17, further comprising selecting a piece of DNA with a desired sequence after the last hierarchical assembly step.

34. The method of claim 33, wherein the selection comprises cloning the piece of DNA with a desired sequence into a frameshift vector.

35. The method of claim 34, wherein the frameshift vector comprises SEQ. ID. NO.: 1 or SEQ. ID. NO.: 2.

36. The method of claim 1, further comprising producing pools of oligonucleotides corresponding to the pools of DNA sequences of the final hierarchical division.

37. The method of claim 36, wherein at least one of the pools of oligonucleotides comprises oligonucleotides bound to a solid-state support.

38. The method of claim 37, wherein the solid-state support comprises beads, an array, or combinations thereof.

39. A system for hierarchically synthesizing a piece of DNA with a desired nucleic acid sequence, the system comprising a plurality of pools of oligonucleotides corresponding to pools of DNA sequences of a final hierarchical division produced by the method of claim 1 performed on a nucleic acid sequence of a piece of DNA with a desired nucleic acid sequence.

40. The system of claim 39, wherein at least one pool of oligonucleotides is disposed in a tube or in a well.

41. The system of claim 39, wherein at least one pool of oligonucleotides is bound to a solid-state support.

42. The system of claim 41, wherein the solid-state support comprises beads, an array, or combinations thereof.

43. The system of claim 39, wherein
the piece of DNA with a desired nucleic acid sequence is a member of a pool comprising a plurality of pieces of DNA with desired nucleic acid sequences, and
the plurality of pools of oligonucleotides correspond to pools of DNA sequences of a final hierarchical division produced by the method of claim 1 performed on the nucleic acid sequences of the plurality of pieces of DNA with desired nucleic acid sequences.

44. The system of claim 39, further comprising polymerase chain reaction primers suitable for isolating at least one piece of DNA with a desired nucleic acid sequence.

45. The system of claim 39, further comprising a frameshift vector.

46. The system of claim 39, further comprising instructions for synthesizing a piece of DNA with a desired nucleic acid sequence from the plurality of pools of oligonucleotides.

47. A system for hierarchically synthesizing a piece of DNA with a desired nucleic acid sequence, the system comprising machine readable media comprising machine readable instructions, which when executed, perform the method of claim 1.

48. The system of claim 47, further comprising a data processing unit operatively coupled to the machine readable media, wherein the data processing unit is operable to execute the machine readable instructions.

49. A plurality of pools of DNA sequences corresponding to a plurality of pools of DNA sequences of a final hierarchical division produced by the method of claim 1 performed on a nucleic acid sequence of a piece of DNA with a desired nucleic acid sequence.

50. The plurality of pools of DNA sequences of claim 49, wherein
the piece of DNA with a desired nucleic acid sequence is a member of a pool comprising a plurality of pieces of DNA with desired nucleic acid sequences, and
the plurality of pools of DNA sequences correspond to pools of DNA sequences of a final hierarchical division produced by the method of claim 1 performed on the nucleic acid sequences of the plurality of pieces of DNA with desired nucleic acid sequences.
